(19) United States
(12) Patent Application Publication
(10) Pub. No.: US 2012/0230991 A1
(43) Pub. Date: Sep. 13, 2012

(54) METHODS AND COMPOUNDS FOR ENHANCING ANTI-CANCER THERAPY

(76) Inventors: Douglas Kim Graham, Aurora, CO (US); Rachel Linger, Denver, CO (US); Deborah Deryckere, Boulder, CO (US); Susan Louise Sather, Denver, CO (US); Amy Keating, Denver, CO (US); Grace Kim, Aurora, CO (US); Luis Brandao, Denver, CO (US)

(21) Appl. No.: 13/055,101

(22) PCT Filed: Jul. 29, 2009
(86) PCT No.: PCT/US09/52160

§ 371 Date: Jul. 28, 2011

Related U.S. Application Data
(60) Provisional application No. 61/084,259, filed on Jul. 29, 2008.

(51) Int. Cl. A61K 39/395; A61P 35/00; C12N 5/09

(52) U.S. Cl. 424/134.1; 424/158.1; 435/375

(57) ABSTRACT

The invention provides methods of treating neoplastic disorders in a mammal through the inhibition of Mer and/or AxI receptor tyrosine kinases as well as compounds and compositions useful for inhibiting these kinases in these methods. These treatment methods may be combined with the administration of one or more chemo therapeutic agent(s) to enhance the efficacy or minimize the toxicities of the chemotherapeutic agent(s).

### TECHNICAL FIELD

The invention relates to the treatment of neoplastic disorders and particularly mammalian cancers through the inhibition of Mer or Axl transmembrane receptor tyrosine kinases alone or synergistically with the administration of other chemotherapeutic agents.

### BACKGROUND OF INVENTION

Drug therapies for many cancers continue to be inadequate, having either limited efficacy, prohibitive toxicities, or in many cases both. Results of standard treatment are poor for all but the most localized cancers, and currently, no single chemotherapy or biologic regimen can be recommended for routine use. Thus, there continues to be a need for new therapies that can effectively treat cancer and keep cancer in remission and increase survival.

In recent years, inhibition of specific cancer-associated tyrosine kinases has emerged as an important approach for cancer therapy. Tyrosine kinases as mediators of cell signaling, play a role in many diverse physiological pathways including cell growth and differentiation. Deregulation of tyrosine kinase activity can result in cellular transformation leading to the development of human cancer. Of the nearly thirty novel cancer targets extensively studied in the past ten years, one third of these are tyrosine or other kinases. Of the ten truly novel anti-cancer therapies approved in the past five years, five have been directed against receptor tyrosine kinases (RTKs). In fact, many cancer treatment protocols now use a combination of traditional chemotherapy drugs and novel biologically targeted agents, several of which inhibit tyrosine kinase activity or downstream signaling pathways. For example, a small molecule drug that inhibits the abl tyrosine kinase has led to significant improvement in outcomes for patients with chronic myelogenous leukemia. Inhibitors of other tyrosine kinases, including the Flt-3, EGFR, and PDGF receptor tyrosine kinases are also in clinical trials.

Mer is a transmembrane receptor tyrosine kinase that is likely the human homologue of the chicken retroviral gene, v-eyk, which causes many types of cancer in chicken. The human Mer gene and the mouse Mer gene and cDNA have been sequenced and characterized, and the expression of Mer has been profiled in cell lines and tissues (Graham et al., Cell Growth and Differentiation, 1994, 5:647-657; Graham et al., Oncogene, 1995, 10:2349-2359; and U.S. Pat. No. 5,585,269). The Mer receptor tyrosine kinase, initially cloned from a human B lymphoblastoid cell line, is expressed in a spectrum of hematopoietic, epithelial, and mesenchymal cell lines. Interestingly, while the RNA transcript of Mer is detected in numerous T and B lymphoblastic cell lines, Mer RNA is not found in normal human thymocytes, lymphocytes or in PMA/PHA stimulated lymphocytes. Mer is composed of two immunoglobulin domains and two fibronectin III domains in the extracellular portion, and a tyrosine kinase domain in the intracellular portion (Graham et al., (1994), supra and Graham et al., (1995), supra). Human Mer is known to be transforming and anti-apoptotic, and Mer overexpression has been linked to a number of different human cancers including subsets of B and T cell leukemia, lymphoma, pituitary adenoma, gastric cancer, and rhabdomyosarcoma. Methods and compounds for inhibiting Mer have been described that can effectively inhibit binding of the Mer ligand to an endogenous Mer receptor tyrosine kinase by at least 50% (PCT/US2005/042724, WO 2006/058202).

The Axl receptor tyrosine kinase (Axl), originally identified as a protein encoded by a transforming gene from primary human myeloid leukemia cells, is overexpressed in a number of different tumor cell types and transforms NIH3T3 fibroblasts (O'Bryan et al., Mol. Cell. Bio. 11:5016-5031 (1991)). Axl signaling has been shown to favor tumor growth through activation of proliferative and anti-apoptotic signaling pathways, as well as through promotion of angiogenesis and tumor invasiveness. Axl is associated with the development and maintenance of various cancers including lung cancer, myeloid leukemia, uterine cancer, ovarian cancer, gliomas, melanoma, prostate cancer, breast cancer, gastric cancer, osteosarcoma, renal cell carcinoma, and thyroid cancer, among others. Furthermore, in some cancer types, particularly non-small cell lung cancer (NSCLC), myeloid leukemia, and gastric cancers, the over-expression of this cell signaling molecule indicates a poor prognosis for the patient. Researchers have found that siRNA knockdown of Axl in NSCLC cell lines reduced invasive capacity of the tumor cells (Holland et al., 2005, Cancer Res. 65:9294-9303). Additional research has shown that expression of a dominant-negative Axl construct decreased brain tumor proliferation and invasion (Vajkoczy et al., 2006, PNAS 15:5799-804; European Patent Publication No. EP 1 382 969 A1). Furthermore, in clinical patient samples of NSCLC, Axl protein over-expression has been statistically associated with lymph node involvement and advanced clinical stage of disease. Methods and compounds for inhibiting Axl have been described that can effectively inhibit binding of the Axl ligand to an endogenous Axl receptor tyrosine kinase by at least 50% (PCT/US08/53337).

Because it is generally the case in cancer therapy that no single agent can successfully treat a patient, new agents continue to be developed and may ultimately be used in combination with other agents to affect the best outcome for patients. Due to the extensive evidence linking the Mer and Axl receptor tyrosine kinases and their evolutionary counterparts to mammalian cancers, as well as the efficacy of treating certain cancers seen with the inhibition of these kinases, there is a recognized need to identify ways of successfully using the inhibition of Mer and Axl tyrosine kinases to effectively target and prevent or treat neoplastic diseases.

### SUMMARY OF INVENTION

The invention relates to treating or preventing neoplastic disorders in a mammal by inhibiting the activity of a receptor tyrosine kinase in the mammal. The receptor tyrosine kinase may be Axl, Mer or Tyro-3 receptor tyrosine kinases (the TAM family of tyrosine receptor kinases).

In one aspect, the neoplastic disorder is a cancer such as glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendothelio sarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyo sarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcorna, chondrosarcoma, osteogenic sarcoma, leukemia and metastatic lesions secondary to these primary tumors. In a related aspect, the cancer is a leukemia or lymphoma and in a preferred embodiment, the cancer is a myeloid leukemia or a lymphoid leukemia or lymphoma. In another preferred embodiment, the cancer is a non-small cell lung cancer (NSCLC). In another preferred embodiment, the cancer is an astrocytoma or gliobalstoma.

In another aspect, the receptor tyrosine kinase is inhibited by the administration of a fusion protein to the mammal. The fusion protein may be composed of at least a portion of the Axl or Mer receptor tyrosine kinase extracellular domain fused to a construct that can bind and aggregate two or more of the receptor tyrosine kinase extracellular domain. In a preferred embodiment, the construct is an Fc region of a human immunoglobulin. The fusion protein may also be composed of at least a portion of the Axl or Mer receptor tyrosine kinase extracellular domain fused to the Fc region of a human immunoglobulin. The fusion protein may also be a combination of a protein that is composed of at least a portion of the Axl receptor tyrosine kinase extracellular domain fused to the Fc region of a human immunoglobulin and a protein that is composed of at least a portion of the Mer receptor tyrosine kinase extracellular domain fused to the Fc region of a human immunoglobulin.

In another aspect, the receptor tyrosine kinase is inhibited by the administration of an antibody that results in the downregulation of at least one of Axl, Mer and Tyro-3 tyrosine kinases to the mammal. In a preferred embodiment, the antibody is a monoclonal antibody that recognizes an epitope on the extracellular domain of the Axl receptor tyrosine kinase. In another preferred embodiment, the antibody is a monoclonal antibody that recognizes an epitope on the extracellular domain of the Mer receptor tyrosine kinase. In another preferred embodiment, the antibody is a monoclonal antibody that recognizes an epitope on the extracellular domain of the Tyro-3 receptor tyrosine kinase. In a related aspect, the receptor tyrosine kinase is inhibited by the administration of at least two antibodies that downregulate of at least one of Axl, Mer and Tyro-3 tyrosine kinases in the mammal.

In another aspect, the receptor tyrosine kinase is inhibited by the administration of a compound that inhibits the kinase activity of the receptor tyrosine kinase protein. In a preferred embodiment, the compound interacts directly with the tyrosine kinase domain of the tyrosine kinase protein to inhibit the kinase activity of the protein. In one embodiment a single compound may be administered that effectively inhibits the kinase activity of at least two of Axl, Mer and/or Tyro-3 receptor tyrosine kinases.

In one aspect of the invention, a chemotherapeutic drug is administered to the mammal just prior to, concurrent with, or immediately following the inhibition of the receptor tyrosine kinase. In this aspect, the inhibition of the receptor tyrosine kinase effectively enhances the sensitivity of neoplastic cells to the chemotherapeutic drug. In one embodiment, this results in increased killing of the neoplastic cells by the chemotherapeutic drug compared to the killing of these cells that results from the administration of the same chemotherapeutic drug in the absence of the inhibition of the receptor tyrosine kinase. In another embodiment, this administration results in overcoming resistance of the neoplastic cells in the mammal to the chemotherapeutic drug, effectively sensitizing the neoplastic cells to the chemotherapeutic drug compared to the sensitivity of these cells to the same chemotherapeutic drug in the absence of the inhibition of the receptor tyrosine kinase. In another embodiment, this administration results in decreasing the toxicity of the chemotherapeutic drug in the mammal by decreasing the dosage of the chemotherapeutic drug that is required to effectively treat the neoplastic disorder in the mammal compared with the dosage required to treat the neoplastic disorder in the mammal in the absence of the inhibition of the receptor tyrosine kinase. In another embodiment, this administration results in a synergistic activity between the inhibition of the receptor tyrosine kinase and the anti-cancer activity of the chemotherapeutic drug that produces an unexpectedly effective treatment of the neoplastic disorder in the mammal compared to the efficacy of the treatment of the neoplastic disorder in the mammal in the presence of either the inhibition of the receptor tyrosine kinases or the chemotherapeutic drug, individually.

In one aspect, the chemotherapeutic drug is administered simultaneously with an inhibitor of the receptor tyrosine kinases. In one embodiment, the chemotherapeutic drug is administered in a composition that contains the drug and at least one agent that inhibits the receptor tyrosine kinase. In another embodiment, the chemotherapeutic drug and an agent that inhibits the receptor tyrosine kinase are administered in separate compositions, simultaneously to the mammal.

In another aspect, the chemotherapeutic drug is administered at a time during the treatment of the neoplastic disorder in the mammal when the tyrosine receptor kinase in the mammal is effectively inhibited compared to the activity of the receptor tyrosine kinase in the mammal in the absence of any inhibition. In one embodiment, the chemotherapeutic drug is administered to the mammal within one week of the inhibition of the receptor tyrosine kinase. In a related embodiment, the chemotherapeutic drug is administered to the mammal within three weeks of the inhibition of the receptor tyrosine kinase. In a preferred embodiment, the receptor tyrosine kinase is effectively inhibited throughout the administration regimen of the chemotherapeutic drug during the treatment of the neoplastic disorder in the mammal.

In one aspect, the chemotherapeutic drug is any one of busulfan, thiotepa, chlorambucil, cyclophosphamide, estramustine, ifosfamide, mechloretharmine, melphalan, uramustine, carmustine, lomustine, streptozocin, dacarbazine, procarbazine, temozolamide, cisplatin, carboplatin, oxaliplatin, satraplatin, picoplatin, methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, fluorouracil, gemcitabine, troxacitabine, bleomycin, dactinomycin, mithramycin, mitomycin, mitoxantrone, porfiromycin, daunorubicin, daunorubicin, doxorubicin, liposomal doxorubicin, epirubicin, idarubicin, valrubicin, L-asparaginase, PEG-L-asparaginase, paclitaxel, docetaxel, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifine, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, lenalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, ¹³¹I-tositumomab, interferon-α₂ₐ, interferon-α_{2b}, aldesleukin, denileukin diftitox, and oprelvekin. In a preferred embodiment, the chemotherapeutic drug is at least one of 6-mercaptopurine, etoposide, adriamycin, vincristine and methotrexate.

In one aspect, the receptor tyrosine kinase is inhibited by contacting the neoplastic cells with an antibody that recognizes an epitope on the extracellular domain of at least one of an Axl and Mer receptor tyrosine kinase. In one embodiment, the antibody is a monoclonal antibody against the Axl receptor tyrosine kinase. In one embodiment, the antibody is a monoclonal antibody against the extracellular domain of the Axl receptor tyrosine kinase. In one embodiment, the antibody is a monoclonal antibody against the Mer receptor tyrosine kinase. In one embodiment, the antibody is a monoclonal antibody against the extracellular domain of the Mer receptor tyrosine kinase.

In one aspect, the receptor tyrosine kinase is inhibited by contacting the neoplastic cells with an shRNA molecule that downregulates an RNA transcript of at least one of an Axl and Mer receptor tyrosine kinase. In one embodiment, the contact with the shRNA molecule results in at least a 50% knockdown of the receptor tyrosine kinase protein in the neoplastic cells. In one embodiment, the contact with the shRNA molecule results in at least a 70% knockdown of the receptor tyrosine kinase protein in the neoplastic cells. In one embodiment, the contact with the shRNA molecule results in at least an 80% knockdown of the receptor tyrosine kinase protein in the neoplastic cells.

In one aspect, the receptor tyrosine kinase is inhibited by contacting the neoplastic cells with an agent that decreases or prevents the binding of a ligand to at least one of an Axl and a Mer receptor tyrosine kinase. In one embodiment, the agent is an antibody that recognizes the ligand. In another embodiment, the agent is an antibody that recognizes an epitope on the Axl or Mer receptor tyrosine kinase. In another embodiment, the agent is a fusion protein composed of at least a portion of the Axl or the Mer receptor tyrosine kinase protein fused to a human immunoglobulin fragment.

Another aspect is a method of preventing a cancer in a mammal by eliminating cancer stem cell populations in the mammal. The cancer stem cells are cells expressing at least one receptor tyrosine kinase of the Axl, Mer or Tyro-3 tyrosine receptor kinases. In one embodiment, the stem cells are eliminated by inducing apoptosis in the stem cells by contacting the cells with an inhibitor of the receptor tyrosine kinase. In another embodiment, the stem cells are eliminated through necrosis of these stem cells following contact of the cells with an inhibitor of the receptor tyrosine kinase. In one embodiment, the stem cells are eliminated by contacting the cells with an antibody that recognizes an epitope on the extracellular domain of at least one of an Axl and Mer receptor tyrosine kinase. In one embodiment, the stem cells are eliminated by contacting the cells with an shRNA molecule that downregulates an RNA transcript of at least one of an Axl and Mer receptor tyrosine kinase. In one embodiment, the stem cells are eliminated by contacting the cells with an inhibitor of the ligand binding of at least one of an Axl and Mer receptor tyrosine kinase. In one embodiment, the stem cells are eliminated by contacting the cells with a fusion protein composed of an Fc region of a human antibody fused with at least a portion of an extracellular domain of Axl receptor tyrosine kinase or at least a portion of an extracellular domain of Mer receptor tyrosine kinase.

Another aspect of the invention is an inhibitor of at least one receptor tyrosine kinase. In one embodiment the inhibitor is an antibody that recognizes an epitope on the extracellular domain of at least one of an Axl and Mer receptor tyrosine kinase. In one embodiment, the inhibitor is an shRNA molecule that downregulates an RNA transcript of at least one of an Axl and Mer receptor tyrosine kinase. In one embodiment, the inhibitor is an inhibitor of the ligand binding of at least one of an Axl and Mer receptor tyrosine kinase. In one embodiment the inhibitor is a fusion protein composed of an Fc region of a human antibody fused with at least a portion of an extracellular domain of Axl receptor tyrosine kinase or at least a portion of an extracellular domain of Mer receptor tyrosine kinase. In one embodiment, the inhibitor is a compound that inhibits the kinase activity of the receptor tyrosine kinase. In a preferred embodiment the inhibitor is a compound that interacts with the tyrosine kinase domain of the receptor tyrosine kinase to inhibit the kinase activity of the receptor.

Another aspect is a composition containing at least one of these receptor tyrosine kinase inhibitors and another therapeutic entity. In one embodiment, the therapeutic entity is a chemotherapeutic drug. In one embodiment the chemotherapeutic drug is at least one of 6-mercaptopurine, etoposide, adriamycin, vincristine and methotrexate. In another aspect, the composition contains at least one agent that modifies the storage stability of the receptor tyrosine kinase in the composition.

Another aspect is a pharmaceutical composition containing at least one receptor tyrosine kinase inhibitor and a pharmaceutically acceptable excipient. In one embodiment, the pharmaceutical composition also contains a chemotherapeutic drug. In one embodiment the chemotherapeutic drug in the pharmaceutical composition is at least one of 6-mercaptopurine, etoposide, adriamycin, vincristine and methotrexate. In another aspect, the pharmaceutical composition contains at least one excipient that that enhances the storage stability of the receptor tyrosine kinase in the composition.

### BRIEF DESCRIPTION OF DRAWINGS

FIG. 1 is a schematic drawing of the Tyro-3, Axl and Mer (TAM) family of receptor tyrosine kinases (RTK) showing the intracellular and extracellular domains of these proteins.

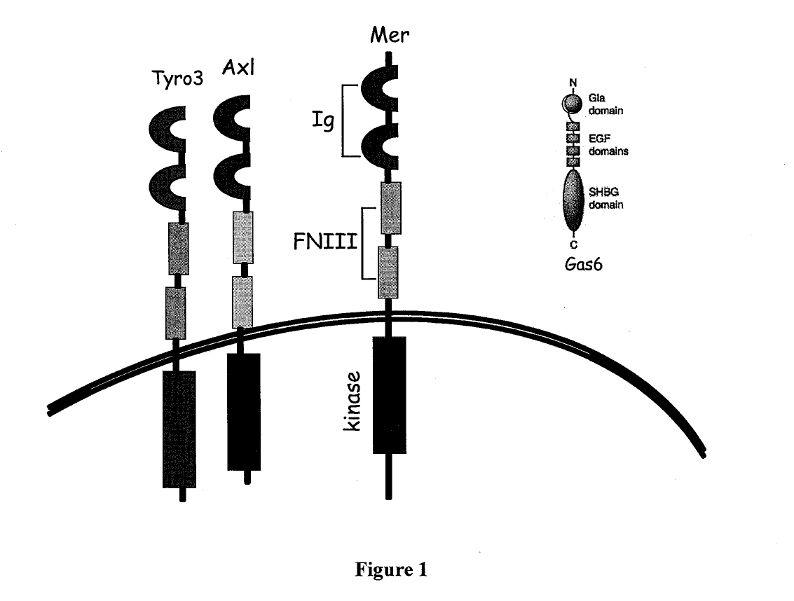

FIGS. 2A, 2B and 2C show Western blot analysis of Mer and Axl protein present in human Acute Lymphoblastic Leukemia (ALL) cells, human lung adenocarcinoma cells, and human glioblastoma cells, respectively.

FIG. 3A shows Western blot analysis of whole cell lysates of E2A-PBX1+Human B-ALL 697 cells infected with lentiviral particles containing short hairpin RNA (shRNA) constructs targeting MerTK or GFP as a non-silencing control. FIG. 3B shows flow cytometry analysis of the cells in FIG. 3A.

FIG. 4 shows a Western blot analysis of whole cell lysates demonstrating Mer expression in glioblastoma stem cell populations, as identified by the CD133 cell marker. Three different glioblastoma patient samples are shown.

FIG. 5 shows wild type, shControl, and Mer knockdown (shMer1A, shMer1B) 697 cells treated with the indicated concentrations of 6-mercaptopurine (6-MP), methotrexate (MTX), vincristine (VCR), etoposide (VP-16), or doxorubicin (DOXO).

FIG. 6 shows cultures of REH wild type, shControl or Mer knockdown (shMer4A, shMer4B) cells treated with methotrexate (MTX) for 48 hours: A) relative cell numbers were determined by MTT assay; B) IC₅₀ values determined by non-linear regression of data from 3 independent experiments performed in triplicate.

FIG. 7 shows cultures of shControl or shMer1A 697 cells exposed to 6-MP, VP-16, or medium only for 48 hours. Apoptotic and dead cells were identified by flow cytometric analysis of cells stained with YO-PRO®-1 and propidium iodide: A) representative flow cytometry profiles; B) mean values and standard errors derived from 3 independent experiments.

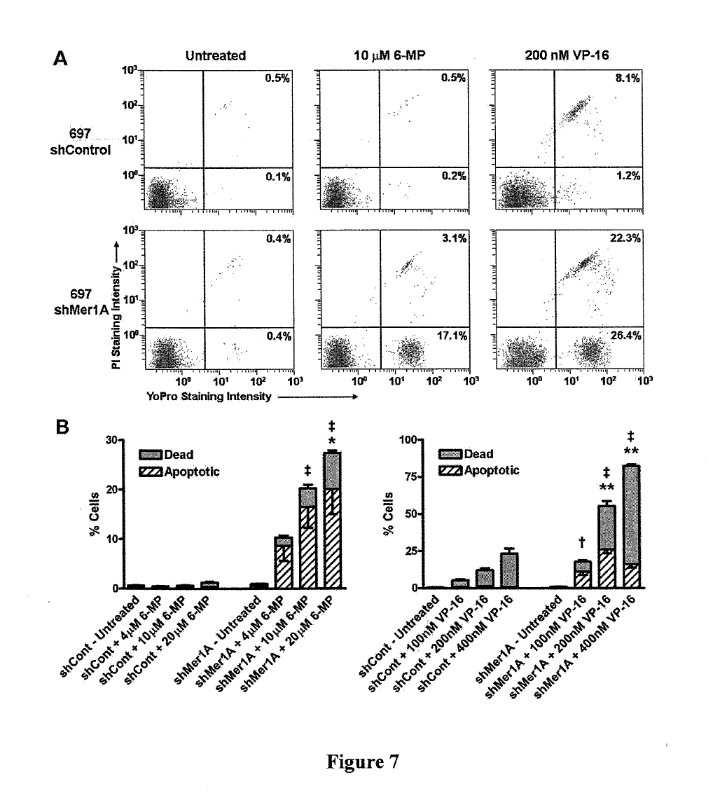

FIG. 8 shows cultures of REH wild type, shControl or Mer knockdown (shMer4A, shMer4B) cells exposed to methotrexate (MTX) or medium only for 48 hours. Apoptotic and dead cells were identified by flow cytometric analysis of cells stained with YO-PRO®-1 and propidium iodide: A) representative flow cytometry profiles. The percentage of apoptotic cells is indicated in the lower-right quadrant. The combined percentage of cells in the two upper quadrants indicates the fraction of dead cells. B) shows the mean values and standard errors derived from 3 independent experiments.

FIG. 9 shows control (shCont) and Mer knockdown (shMer1A) 697 cells exposed to 10 μM 6-MP, 150 nM VP-16, or medium only (Untrt) for 24 hours (A) or 40-60 minutes (B). Whole cell lysates were prepared and expression of the indicated proteins (p− denotes a phosphorylated protein) was determined by western blot analysis. Blots representative of three independent experiments are shown.

FIG. 10 shows NOD-SCID mice injected with cells of the indicated cell lines: A) sub-lethally irradiated NOD-SCID and B) non-irradiated NOD-SCID mice. Ticks on the Kaplan-Meier survival curves indicate censored subjects (mice for which samples could not be obtained or did not have leukemia at time of death).

FIG. 11 shows the relative cell numbers of wild type, shControl, and Axl knockdown (shAxl8-G5) A549 cells treated with the indicated concentrations of (A) cisplatin, (B) carboplatin, (C) doxorubicin, or (D) etoposide for 48 hours.

FIG. 12 shows relative cell proliferation of wild type, shControl, and Mer knockdown (Mer1-G8) A549 cells treated with the indicated concentrations of (A) cisplatin, (B) carboplatin, (C) doxorubicin, or (D) etoposide.

FIG. 13 shows increased induction of apoptosis in response to treatment with doxorubicin (DOXO) and the survival of untreated A549 cells with knockdown of Mer.

FIG. 14 shows the chemosensitivity of astrocytoma cells as evaluated by MTT assay: A) G12 control (shControl) and knockdown cells (shMer1 and shMer4; shAxl8 and shAxl9) plated in 96-well plates and treated with varying concentrations of temozolomide; B) A172 control (shControl) and knockdown cells (shMer1A and shMer1B; shAxl8 and shAxl9) plated in 96-well plates and treated with varying concentrations of carboplatin.

FIG. 15 shows the downregulation of Mer on the surface of 697 leukemia cells after treatment with Mer monoclonal antibody: A) SDS-PAGE of whole cell lysates following treatment of 697 cells with either 5 mg Mer monoclonal antibody or no antibody; and B) synergy between Mer monoclonal antibody treatment and 6-MP in 697 leukemia cell line to decrease ability of leukemia cells to proliferate.

### DESCRIPTION OF EMBODIMENTS

The present invention is drawn to methods of enhancing the efficacy of the chemotherapeutic treatment of a mammal by inhibiting at least one of the Mer and Axl tyrosine kinases in the mammal. The invention also provides compounds and compositions that are useful in the methods of inhibiting these kinases. These methods result in a synergism that extends beyond the efficacy of many well known chemotherapeutic regimens.

### Methods of the Invention

The present invention relates to methods of treatment (prophylactic and/or therapeutic) for Axl-positive cancers, Mer-positive cancers and Tyro-3-positive cancers using Axl inhibitors, Mer inhibitors, chemotherapeutic drugs and combinations of these therapeutic entities. The methods of use of the inhibitors and therapeutic compositions of the present invention preferably provides a benefit to a patient or individual by inhibiting at least one biological activity of Axl and/or Mer or their related receptor Tyro-3.

As used herein, “treatment” refers to clinical intervention in an attempt to alter the natural course of the individual or cell being treated, and may be performed either for prophylaxis and/or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis. Accordingly, a therapeutic benefit is not necessarily a cure for a particular disease or condition, but rather, preferably encompasses a result which most typically includes alleviation of the disease or condition, elimination of the disease or condition, reduction of a symptom associated with the disease or condition, prevention or alleviation of a secondary disease or condition resulting from the occurrence of a primary disease or condition (e.g., metastatic tumor growth resulting from a primary cancer), and/or prevention of the disease or condition.

In the case of cancer, the method of the invention increases the death of tumor cells, decreases the invasive potential of tumor cells, increases the survival of an individual with cancer, and/or increases tumor regression, decreases tumor growth, and/or decreases tumor burden in the individual.

A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

According to the present invention, the methods and assays disclosed herein are suitable for use in or with regard to an individual that is a member of the Vertebrate class, Mammalia, including, without limitation, primates, livestock and domestic pets (e.g., a companion animal). Most typically, a patient will be a human patient. According to the present invention, the terms “patient,” “individual” and “subject” can be used interchangeably, and do not necessarily refer to an animal or person who is ill or sick (i.e., the terms can reference a healthy individual or an individual who is not experiencing any symptoms of a disease or condition).

Diseases and disorders that are characterized by altered (relative to a subject not suffering from the disease or disorder) Axl receptor tyrosine kinases, Mer receptor tyrosine kinases, levels of these proteins, and/or biological activity associated with these proteins, are treated with therapeutics that antagonize (e.g., reduce or inhibit) the Axl and/or Mer receptor tyrosine kinases or their ligands. The therapeutic entities of the present invention block the activation of the full length native Axl and/or Mer receptor kinases by binding to Axl and/or Mer ligands including, but necessarily limited to, Gas6. Therefore, an effective amount of an inhibitor of a Gas6 receptor which is provided in the form of an Axl or Mer inhibitor described herein may be used as a treatment for diseases and conditions associated with Axl or Mer expression, as well as with Tyro-3 expression.

Accordingly, the method of the present invention preferably modulates the activity of Axl and/or Mer receptor tyrosine kinases, thereby increasing the sensitivity of these cells to the effects of a chemotherapeutic drug.

A chemotherapeutic drug may be any chemical administered to a mammal for the purpose of killing, arresting the development, preventing or slowing the metastasis or inducing the regression of neoplastic cells in the mammal. As used herein, the terms “neoplasm,” “tumor” or “cancer” refers to any neoplastic disorder, including carcinomas, sarcomas and carcino-sarcomas. Specific types of neoplastic disorders include, without limitation, glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyo sarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcorna, chondrosarcoma, osteogenic sarcoma, leukemia, and the metastatic lesions secondary to these primary tumors. In general, any neoplastic lesion, including granulomas, may be included according the present invention. Therefore, the “cancer cells” in this invention also includes the cancer-supporting components such as tumor endothelial cells. The composition and method of the invention are useful for the treatment of not only any cancer in which Axl or Mer are expressed, but also any cancer in which Tyro-3 is expressed. In preferred embodiments, the cancers that may be treated using the methods of the present invention include lung cancer (including, but not limited, to non-small cell lung cancer), myeloid leukemia, uterine cancer, ovarian cancer, gliomas, melanoma, prostate cancer, breast cancer, gastric cancer, colon cancer, osteosarcoma, renal cell carcinoma, and thyroid cancer. In one aspect, the cancer is selected from any one of lung cancer, myeloid leukemia, uterine cancer, ovarian cancer, gliomas, melanoma, prostate cancer, breast cancer, gastric cancer, osteosarcoma, renal cell carcinoma, or thyroid cancer. In one preferred aspect, the cancer is a leukemia or lymphoma. In another preferred aspect, the cancer is myeloid leukemia. In another preferred aspect, the cancer is non-small cell lung cancer (NSCLC).

Exemplary chemotherapeutic drugs of the invention may include one or more of busulfan, thiotepa, chlorambucil, cyclophosphamide, estramustine, ifosfamide, mechloretharmine, melphalan, uramustine, carmustine, lomustine, streptozocin, dacarbazine, procarbazine, temozolamide, cisplatin, carboplatin, oxaliplatin, satraplatin, picoplatin, methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, fluorouracil, gemcitabine, troxacitabine, bleomycin, dactinomycin, mithramycin, mitomycin, mitoxantrone, porfiromycin, daunorubicin, daunorubicin, doxorubicin, liposomal doxorubicin, epirubicin, idarubicin, valrubicin, L-asparaginase, PEG-L-asparaginase, paclitaxel, docetaxel, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifine, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, lenalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, ¹³¹I-tositumomab, interferon-α₂ₐ, interferon-α_{2b}, aldesleukin, denileukin diftitox, and oprelvekin. In a preferred embodiment, the chemotherapeutic agent is selected from 6-mercaptopurine, etoposide, adriamycin, vincristine and methotrexate.

The method of the invention for example, involves contacting a cell, tissue or system of a mammal with an inhibitor that modulates one or more of the activities of Axl and/or Mer. The Axl/Mer inhibitors act as competitive inhibitors of Axl and/or Mer receptors expressed by cells. Such methods are preferably performed in vivo (e.g., by administering the agent to a subject). As such, the invention provides methods of treating an individual afflicted with a disease or disorder or suspected of having a neoplastic disorder, specifically a cancer.

As discussed above, and as described and demonstrated in Example 5, infra, Axl and Mer signaling favor tumor growth through activation of proliferative and anti-apoptotic signaling pathways, as well as through promotion of angiogenesis and tumor invasiveness. Accordingly, it is another embodiment of the present invention to inhibit Axl or Mer activity as part of a therapeutic strategy which selectively targets cancer cells. Inhibition is also provided by the present invention in this embodiment through the administration of the Axl and/or Mer inhibitor(s) described herein (e.g., Axl-Fc, Mer-Fc), which bind directly to Axl ligands and competitively inhibit the binding of such ligands to Axl, Mer, or Tyro-3, and therefore inhibit the activity of such receptors. The inhibitors may be administered alone or together with a chemotherapeutic agent. In one embodiment, an Axl inhibitor is administered together with a Mer-Fc protein. In some instances, the Mer-Fc protein may cause a transient activation of the Axl receptor tyrosine kinase that may last between about 30 minutes to about 1 hour. Following this temporary activation, both Mer and Axl tyrosine kinase receptors are inhibited for many days or weeks. This transient activation of any of the TAM family receptor tyrosine kinases does not preclude their effective use as inhibitors in the methods of the current invention.

In the therapeutic methods of the invention, suitable methods of administering a composition of the present invention to a subject include any route of in vivo administration that is suitable for delivering the composition. The preferred routes of administration will be apparent to those of skill in the art, depending on the type of delivery vehicle used, the target cell population, and the disease or condition experienced by the patient.

A preferred single dose of a protein such as an Axl and/or Mer inhibitor of the invention typically comprises between about 0.01 microgram×kilogram⁻¹ and about 10 milligram×kilogram⁻¹ body weight of an animal. A more preferred single dose of such an agent comprises between about 1 microgram×kilogram⁻¹ and about 10 milligram×kilogram⁻¹ body weight of an animal. An even more preferred single dose of an agent comprises between about 5 microgram×kilogram⁻¹ and about 7 milligram×kilogram⁻¹ body weight of an animal. An even more preferred single dose of an agent comprises between about 10 microgram×kilogram⁻¹ and about 5 milligram×kilogram⁻¹ body weight of an animal. Another particularly preferred single dose of an agent comprises between about 0.1 microgram×kilogram⁻¹ and about 10 microgram×kilogram⁻¹ body weight of an animal, if the agent is delivered parenterally.

The chemotherapeutic drugs used in the application of the methods of the invention may be administered concurrently with the Axl or Mer inhibitory compounds as a single dosage form incorporating at least one Axl or Mer inhibitory compound and at least one chemotherapeutic drug, or as separate dosage formulations, each containing a component of the combination therapies of the present invention. The Axl or Mer inhibitory compound may be administered to a patient simultaneously with a chemotherapeutic drug or at a time during the treatment of the patient's neoplastic disorder such that one or more neoplastic cells in the patient are sensitized to at least one chemotherapeutic drug administered before or after the administration of the Axl or Mer inhibitory compound. In one embodiment, the Axl or Mer inhibitory compound is an Axl monoclonal antibody or a Mer monoclonal antibody or a combination of these antibodies that are administered once a week or once every other week during a course of treating a patient with at least one chemotherapeutic drug.

### Compounds of Invention

Mer and/or Axl inhibitory compounds of the invention include any biologic or chemical entities that inhibit the activity of the TAM tyrosine receptor kinase family of proteins. The inhibition may include direct or indirect inhibition of one or more of these kinases, down regulation of one or more of these kinases, inhibition or prevention of translation of the Axl or Mer kinase transcripts, inhibition or prevention of post-translation protein modifications of the Axl or Mer kinase proteins, or the production of dysfunctional Axl or Mer transcripts or proteins. Exemplary Axl and Mer kinase inhibitors are described in U.S. patent application Ser. No. 11/720,185, filed May 24, 2007, PCT Application No. PCT/US05/042724 (WO 2006/058202) and PCT Application No. PCT/US08/53337. In a preferred embodiment, the Axl or Mer inhibitory compounds of the invention are Axl or Mer fusion proteins.

One embodiment of the invention relates to an Axl inhibitor, wherein the Axl inhibitor is preferably an Axl fusion protein. The Axl fusion protein contains: (a) a first protein comprising, consisting essentially of, or consisting of, at least a portion of the extracellular domain of an Axl receptor tyrosine kinase (Axl RTK) that binds to an Axl ligand; and (b) a second protein that is a heterologous fusion protein, wherein the second protein is fused to the first protein.

In one aspect, the first protein comprises, consists essentially of, or consists of the Gas6 major binding site of Axl. In one aspect, the first protein comprises, consists essentially of, or consists of the Gas6 major binding site and the Gas6 minor binding site of Axl. In one aspect, the first protein comprises, consists essentially of, or consists of the Ig1 domain of Axl. In one aspect, the first protein comprises, consists essentially of, or consists of the Ig1 domain and the Ig2 domain of Axl. In one aspect, the first protein comprises, consists essentially of, or consists of a portion of the extracellular domain of Axl RTK in which at least one of the FBNIII motifs in the first protein is deleted or mutated of Axl. In one aspect, the first protein comprises, consists essentially of, or consists of a portion of the extracellular domain of Axl RTK in which both of the FBNIII motifs is deleted or mutated of Axl. In one aspect, the first protein comprises, consists essentially of, or consists of, the entire Axl RTK extracellular domain of Axl. In one aspect, the first protein comprises, consists essentially of, or consists of positions 1-445 of Axl RTK, with respect to SEQ ID NO:1. In one aspect, the first protein comprises, consists essentially of, or consists of positions 1-324 or 1-325 of Axl RTK, with respect to SEQ ID NO:1. In one aspect, the first protein comprises, consists essentially of, or consists of position 1 to position 222, 223, 224, or 225 of Axl RTK, with respect to SEQ ID NO:1. In one aspect, the first protein comprises, consists essentially of, or consists of at least: position 10 to position 222, 223, 224, or 225 of Axl RTK, position 20 to position 222, 223, 224, or 225 of Axl RTK, position 30 to position 222, 223, 224, or 225 of Axl RTK, position 40 to position 222, 223, 224, or 225 of Axl RTK, position 50 to position 222, 223, 224, or 225 of Axl RTK, or position 60 to position 222, 223, 224, or 225 of Axl RTK, with respect to SEQ ID NO:1. In one aspect, the first protein comprises, consists essentially of, or consists of: at least positions 63-225 of SEQ ID NO:1. In one aspect, the first protein comprises, consists essentially of, or consists of at least: positions 1-137 of Axl RTK, positions 10-137 of Axl RTK, positions 20-137 of Axl RTK, positions 30-137 of Axl RTK, positions 40-137 of Axl RTK, positions 50-137 of Axl RTK, or positions 60-137 or Axl RTK, with respect to SEQ ID NO:1. In one aspect, the first protein comprises, consists essentially of, or consists of at least positions 63 to 218 of SEQ ID NO:1. In one aspect, the first protein comprises at least positions 63-99, 136, 138, and 211-218 of SEQ ID NO:1, arranged in a conformation that retains the tertiary structure of these positions with respect to the full-length extracellular domain of Axl RTK (positions 1-445 of SEQ ID NO:1).

In any of the above aspects of the invention, the Axl RTK can comprise an amino acid sequence that is at least 80% identical, at least 90% identical, or at least 95% identical, to SEQ ID NO:1. In one aspect, the Axl RTK comprises an amino acid sequence of SEQ ID NO:1.

In any of the above aspects of the invention, the fusion protein can be produced as a heterologous fusion protein combining at least a portion of the Axl RTK extracellular domain fused with an immunoglobulin Fc domain. In one aspect, the immunoglobulin Fc domain consists essentially of or consists of a heavy chain hinge region, a CH₂ domain and a CH₃ domain. In one aspect, the immunoglobulin Fc domain is from an IgG immunoglobulin protein. In one aspect, the immunoglobulin Fc domain is from an IgG1 immunoglobulin protein. In one aspect, the immunoglobulin Fc domain is from a human immunoglobulin.

In the aspects of the invention related to an Axl fusion protein, the Axl fusion protein binds to the Axl ligand with an equal or greater affinity when compared to a naturally occurring Axl receptor tyrosine kinase. In one aspect, the Axl fusion protein inhibits binding of the Axl ligand to an endogenous Axl receptor tyrosine kinase by at least 50%. In another aspect, the Axl fusion protein inhibits binding of the Axl ligand to an endogenous Axl receptor tyrosine kinase by at least 60%. In another aspect, the Axl fusion protein inhibits binding of the Axl ligand to an endogenous Axl receptor tyrosine kinase by at least 70%. In another aspect, the Axl fusion protein inhibits binding of the Axl ligand to an endogenous Axl receptor tyrosine kinase by at least 80%.

Another embodiment of the invention relates to a Mer inhibitor, wherein the Mer inhibitor is preferably a Mer fusion protein. The Mer fusion protein contains: (a) a first protein comprising, consisting essentially of, or consisting of, at least a portion of the extracellular domain of a Mer receptor tyrosine kinase (Mer RTK) that binds to a Mer ligand; and (b) a second protein that is a heterologous fusion protein, wherein the second protein is fused to the first protein.

In one aspect, the first protein comprises, consists essentially of, or consists of the Gas6 major binding site of Mer. In one aspect, the first protein comprises, consists essentially of, or consists of the Gas6 major binding site and the Gas6 minor binding site of Mer. In one aspect, the first protein comprises, consists essentially of, or consists of the Ig1 domain of Mer. In one aspect, the first protein comprises, consists essentially of, or consists of the Ig1 domain and the Ig2 domain of Mer. In one aspect, the first protein comprises, a Mer protein consisting of the Mer extracellular domain (e.g., positions 1 to about 473 of SEQ ID NO:2), or smaller portions of this extracellular domain that retain the ability to bind to at least one Mer ligand, fused to the second protein.

In any of the above aspects of the invention, the Mer RTK can comprise an amino acid sequence that is at least 80% identical, at least 90% identical, or at least 95% identical, to SEQ ID NO:2. In one aspect, the Mer RTK comprises an amino acid sequence of SEQ ID NO:2.

In any of the aspects of the invention related to a Mer fusion protein, the second protein that is a heterologous fusion protein can be produced by combining at least a portion of the Mer RTK extracellular domain fused with an immunoglobulin Fc domain. In one aspect, the immunoglobulin Fc domain consists essentially of, or consists of a heavy chain hinge region, a CH₂ domain and a CH₃ domain. In one aspect, the immunoglobulin Fc domain is from an IgG immunoglobulin protein. In one aspect, the immunoglobulin Fc domain is from an IgG1 immunoglobulin protein. In one aspect, the immunoglobulin Fc domain is from a human immunoglobulin.

In any of the aspects of the invention related to a Mer fusion protein, the Mer fusion protein binds to the Mer ligand with an equal or greater affinity as compared to a naturally occurring Mer receptor tyrosine kinase. In one aspect, the Mer fusion protein inhibits binding of the Mer ligand to an endogenous Mer receptor tyrosine kinase by at least 50%. In another aspect, the Mer fusion protein inhibits binding of the Mer ligand to an endogenous Mer receptor tyrosine kinase by at least 60%. In another aspect, the Mer fusion protein inhibits binding of the Mer ligand to an endogenous Mer receptor tyrosine kinase by at least 70%. In another aspect, the Mer fusion protein inhibits binding of the Mer ligand to an endogenous Mer receptor tyrosine kinase by at least 80%.

Fragments within any of these specifically defined Axl or Mer fragments are encompassed by the invention, provided that, in one embodiment, the fragments retain ligand binding ability of Axl or Mer, preferably with an affinity sufficient to compete with the binding of the ligand to its natural receptor (e.g., naturally occurring Axl or Mer) and provide inhibition of a biological activity of Axl or Mer or provide a therapeutic benefit to a patient. It will be apparent that, based on the knowledge of residues important for binding to Gas6 within these regions, various conservative or even non-conservative amino acid substitutions can be made, while the ability to bind to Gas6 is retained. Fragments within any of the above-defined fragments are also encompassed by the invention if they additionally (ligand binding also required), or alternatively (ligand binding not retained), retain the ability to bind to a TAM receptor (at least one TAM receptor binding domain) sufficient to inhibit activation and signaling through the TAM receptor (e.g., by preventing/blocking ligand binding or by preventing receptor dimerization, trimerization or formation of any receptor-protein complex).

Assays for measuring binding affinities are well-known in the art. In one embodiment, a BIAcore machine can be used to determine the binding constant of a complex between the target protein (e.g., an Axl-Fc or a Mer-Fc) and a natural ligand (e.g., Gas6). For example, the Axl or Mer inhibitor can be immobilized on a substrate. A natural or synthetic ligand is contacted with the substrate to form a complex. The dissociation constant for the complex can be determined by monitoring changes in the refractive index with respect to time as buffer is passed over the chip (O'Shannessy et al. Anal. Biochem. 212:457-468 (1993); Schuster et al., Nature 365:343-347 (1993)). Contacting a second compound (e.g., a different ligand or a different Axl or Mer protein) at various concentrations at the same time as the first ligand and monitoring the response function (e.g., the change in the refractive index with respect to time) allows the complex dissociation constant to be determined in the presence of the second compound and indicates whether the second compound is an inhibitor of the complex. Other suitable assays for measuring the binding of a receptor to a ligand include, but are not limited to, Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

In one embodiment, all or a portion of the extracellular domain of Axl or Mer can be deleted or mutated. Again, any deletions or other mutations (substitutions, additions, etc.) are encompassed by the invention, provided that the ligand-binding ability of the Axl- or Mer-containing protein is retained.

According to the present invention, an Fc protein or fragment (also referred to as Fc domain or Fc region of an immunoglobulin) is a portion of an immunoglobulin (also referred to herein as antibody) lacking the ability to bind to antigen. More particularly, the Fc region (from “Fragment, crystallizable”) of an immunoglobulin, is derived from the constant region domains of an immunoglobulin and is generally composed of two heavy (H) chains that each contribute between two and three constant domains (depending on the isotype class of the antibody), also referred to as C_{H} domains. The Fc region, as used herein, preferably includes the “hinge” region of an immunoglobulin, which joins the two heavy (H) chains to each other via disulfide bonds. Alternatively, if the hinge region is not included, then the Fc region is designed with a region that otherwise links the two heavy chains together, since the Axl-Fc protein may be produced as a dimer of Axl extracellular domains (U.S. Pat. No. 6,323,323 may be reviewed for a generic description of a method for producing dimerized polypeptides).

There are five major H chain classes referred to as isotypes, and accordingly, an Fc protein used in the present invention may be derived from any one of these five classes. The five classes include immunoglobulin M (IgM or μ), immunoglobulin D (IgD or 6), immunoglobulin G (IgG or γ), immunoglobulin A (IgA or α), and immunoglobulin E (IgE or ε). The distinctive characteristics between such isotypes are defined by the constant domain of the immunoglobulin. Human immunoglobulin molecules comprise nine isotypes, IgM, IgD, IgE, four subclasses of IgG including IgG1 (γ1), IgG2 (γ2), IgG3 (γ3) and IgG4 (γ4), and two subclasses of IgA including IgA1 (α1) and IgA2 (α2). The nucleic acid and amino acid sequences of immunoglobulin proteins and domains, including from all isotypes, are well-known in the art in a variety of vertebrate species. Preferably, the Fc region used in the Axl-Fc and Mer-Fc proteins is from the same animal species as the Axl or Mer portion of the protein and most preferably, is from the same animal species as the animal species in which the Axl-Fc or Mer-Fc protein is to be used in vivo. For example, for use in humans, it is preferred that a human Axl or Mer protein and a human Fc protein are fused. However, to the extent that Axl or Mer from one species will bind Gas6 from a different species and may be tolerated for use in such species, such cross-use is encompassed by the invention.

Fc regions used in the fusion proteins of the present invention include any Fc region. Preferred Fc regions include the hinge region and the CH₂ and CH₃ domains of IgG, and preferably, IgG1, although Fc regions of other immunoglobulins can be used. Preferably, the Fc protein does not interfere with the ability of the fusion protein to remain soluble and circulate in vivo, and does not interfere with the ability of the Axl or Mer portion to bind to its respective ligand. As discussed above, a suitable Fc protein may or may not include the hinge region of the immunoglobulin, but if not, should be otherwise capable of being linked to another Fc protein so that the Axl portion of the fusion protein can be expressed as a dimer.

Accordingly, general embodiments of the present invention pertain to any isolated polypeptides described herein, including various portions of full-length Axl, and including those expressed by nucleic acids encoding Axl or a portion or variant thereof.

As used herein, reference to an isolated protein or polypeptide in the present invention, including an isolated Axl or Mer protein, includes full-length proteins, fusion proteins, or any fragment or other homologue (variant) of such a protein. Reference to an Axl or a Mer protein can include, but is not limited to, purified Axl or Mer proteins, recombinantly produced Axl or Mer proteins, membrane bound Axl or Mer proteins, Axl or Mer proteins complexed with lipids, soluble Axl or Mer proteins, an Axl or Mer fusion protein, a biologically active homologue of an Axl or Mer protein, and an isolated Axl or Mer protein associated with other proteins. More specifically, an isolated protein, such as an Axl or Mer protein, according to the present invention, is a protein (including a polypeptide or peptide) that has been removed from its natural milieu (i.e., that has been subject to human manipulation) and can include purified proteins, partially purified proteins, recombinantly produced proteins, and synthetically produced proteins, for example. As such, “isolated” does not reflect the extent to which the protein has been purified. The term “polypeptide” refers to a polymer of amino acids, and not to a specific length; thus, peptides, oligopeptides and proteins are included within the definition of a polypeptide. As used herein, a polypeptide is said to be “purified” when it is substantially free of cellular material when it is isolated from recombinant and non-recombinant cells, or free of chemical precursors or other chemicals when it is chemically synthesized. A polypeptide, however, can be joined to another polypeptide with which it is not normally associated in a cell (e.g., in a “fusion protein”) and still be “isolated” or “purified.”

In addition, and by way of example, a “human Axl protein” refers to a Axl protein (generally including a homologue of a naturally occurring Axl protein) from a human (Homo sapiens) or to an Axl protein that has been otherwise produced from the knowledge of the structure (e.g., sequence) and perhaps the function of a naturally occurring Axl protein from Homo sapiens. In other words, a human Axl protein includes any Axl protein that has substantially similar structure and function of a naturally occurring Axl protein from Homo sapiens or that is a biologically active (i.e., has biological activity) homologue of a naturally occurring Axl protein from Homo sapiens. As such, a human Axl protein can include purified, partially purified, recombinant, mutated/modified and synthetic proteins.

In addition, and by way of example, a “human Mer protein” refers to a Mer protein (generally including a homologue of a naturally occurring Mer protein) from a human (Homo sapiens) or to a Mer protein that has been otherwise produced from the knowledge of the structure (e.g., sequence) and perhaps the function of a naturally occurring Mer protein from Homo sapiens. In other words, a human Mer protein includes any Mer protein that has substantially similar structure and function of a naturally occurring Mer protein from Homo sapiens or that is a biologically active (i.e., has biological activity) homologue of a naturally occurring Mer protein from Homo sapiens. As such, a human Mer protein can include purified, partially purified, recombinant, mutated/modified and synthetic proteins.

According to the present invention, the terms “modification” and “mutation” can be used interchangeably, particularly with regard to the modifications/mutations to the amino acid sequence of Axl or Mer described herein. An isolated protein useful as an antagonist or agonist according to the present invention can be isolated from its natural source, produced recombinantly or produced synthetically.

The polypeptides of the invention also encompass fragment and sequence variants, generally referred to herein as homologues. As used herein, the term “homologue” is used to refer to a protein or peptide which differs from a naturally occurring protein or peptide (i.e., the “prototype” or “wild-type” protein) by minor modifications to the naturally occurring protein or peptide, but which maintains the basic protein and side chain structure of the naturally occurring form. Such changes include, but are not limited to: changes in one or a few amino acid side chains; changes one or a few amino acids, including deletions (e.g., a truncated version of the protein or peptide) insertions and/or substitutions; changes in stereochemistry of one or a few atoms; and/or minor derivatizations, including but not limited to: methylation, glycosylation, phosphorylation, acetylation, myristoylation, prenylation, palmitation, amidation and/or addition of glycosylphosphatidyl inositol. A homologue can have enhanced, decreased, or substantially similar properties as compared to the naturally occurring protein or peptide. A homologue can include an agonist of a protein or an antagonist of a protein. A homologue of a human Axl protein can include a non-human Axl or Mer protein (i.e., an Axl or Mer protein from a different species).

Variants or homologues include a substantially homologous polypeptide encoded by the same genetic locus in an organism, i.e., an allelic variant, as well as other splicing variants. A naturally occurring allelic variant of a nucleic acid encoding a protein is a gene that occurs at essentially the same locus (or loci) in the genome as the gene which encodes such protein, but which, due to natural variations caused by, for example, mutation or recombination, has a similar but not identical sequence. Allelic variants typically encode proteins having similar activity to that of the protein encoded by the gene to which they are being compared. One class of allelic variants can encode the same protein but have different nucleic acid sequences due to the degeneracy of the genetic code. Allelic variants can also comprise alterations in the 5′ or 3′ untranslated regions of the gene (e.g., in regulatory control regions). Allelic variants are well known to those skilled in the art.

The terms variant or homologue may also encompass polypeptides derived from other genetic loci in an organism, but having substantial homology to any of the previously defined soluble forms of the extracellular Axl receptor tyrosine kinase, or polymorphic variants thereof. Variants also include polypeptides substantially homologous or identical to these polypeptides but derived from another organism. Variants also include polypeptides that are substantially homologous or identical to these polypeptides that are produced by chemical synthesis.

In one embodiment, an Axl homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring Axl amino acid sequence or to any of the extracellular fragments of a naturally occurring Axl amino acid sequence as described herein. A homologue of Axl differs from a reference (e.g., wild-type) Axl protein and therefore is less than 100% identical to the reference Axl at the amino acid level.

In another embodiment, a Mer homologue comprises, consists essentially of, or consists of, an amino acid sequence that is at least about 45%, or at least about 50%, or at least about 55%, or at least about 60%, or at least about 65%, or at least about 70%, or at least about 75%, or at least about 80%, or at least about 85%, or at least about 90%, or at least about 95% identical, or at least about 95% identical, or at least about 96% identical, or at least about 97% identical, or at least about 98% identical, or at least about 99% identical (or any percent identity between 45% and 99%, in whole integer increments), to a naturally occurring Mer amino acid sequence or to any of the extracellular fragments of a naturally occurring Mer amino acid sequence as described herein. A homologue of Mer differs from a reference (e.g., wild-type) Mer protein and therefore is less than 100% identical to the reference Mer at the amino acid level.

As used herein, unless otherwise specified, reference to a percent (%) identity refers to an evaluation of homology which is performed using: (1) a BLAST 2.0 Basic BLAST homology search using blastp for amino acid searches and blastn for nucleic acid searches with standard default parameters, wherein the query sequence is filtered for low complexity regions by default (described in Altschul, S. F., Madden, T. L., Schääffer, A. A., Zhang, J., Zhang, Z., Miller, W. & Lipman, D. J. (1997) “Gapped BLAST and PSI-BLAST: a new generation of protein database search programs.” Nucleic Acids Res. 25:3389-3402, incorporated herein by reference in its entirety); (2) a BLAST 2 alignment (using the parameters described below); (3) and/or PSI-BLAST with the standard default parameters (Position-Specific Iterated BLAST. It is noted that due to some differences in the standard parameters between BLAST 2.0 Basic BLAST and BLAST 2, two specific sequences might be recognized as having significant homology using the BLAST 2 program, whereas a search performed in BLAST 2.0 Basic BLAST using one of the sequences as the query sequence may not identify the second sequence in the top matches. In addition, PSI-BLAST provides an automated, easy-to-use version of a “profile” search, which is a sensitive way to look for sequence homologues. The program first performs a gapped BLAST database search. The PSI-BLAST program uses the information from any significant alignments returned to construct a position-specific score matrix, which replaces the query sequence for the next round of database searching. Therefore, it is to be understood that percent identity can be determined by using any one of these programs.

Two specific sequences can be aligned to one another using BLAST 2 sequence as described in Tatusova and Madden, (1999), “Blast 2 sequences—a new tool for comparing protein and nucleotide sequences,” FEMS Microbiol Lett. 174:247-250. BLAST 2 sequence alignment is performed in blastp or blastn using the BLAST 2.0 algorithm to perform a Gapped BLAST search (BLAST 2.0) between the two sequences allowing for the introduction of gaps (deletions and insertions) in the resulting alignment. For purposes of clarity herein, a BLAST 2 sequence alignment is performed using the standard default parameters as follows.

For blastn, using 0 BLOSUM62 matrix:

Reward for match=1

Penalty for mismatch=−2

Open gap (5) and extension gap (2) penalties

gap x_dropoff (50) expect (10) word size (11) filter (on)

For blastp, using 0 BLOSUM62 matrix:

Open gap (11) and extension gap (1) penalties

gap x_dropoff (50) expect (10) word size (3) filter (on).

In one embodiment of the present invention, any of the amino acid sequences described herein, including homologues of such sequences (e.g., Axl or Mer extracellular domains), can be produced with from at least one, and up to about 20, additional heterologous amino acids flanking each of the C- and/or N-terminal end of the given amino acid sequence. The resulting protein or polypeptide can be referred to as “consisting essentially of” a given amino acid sequence. According to the present invention, the heterologous amino acids are a sequence of amino acids that are not naturally found (i.e., not found in nature, in vivo) flanking the given amino acid sequence or which would not be encoded by the nucleotides that flank the naturally occurring nucleic acid sequence encoding the given amino acid sequence as it occurs in the gene, if such nucleotides in the naturally occurring sequence were translated using standard codon usage for the organism from which the given amino acid sequence is derived. Similarly, the phrase “consisting essentially of,” when used with reference to a nucleic acid sequence herein, refers to a nucleic acid sequence encoding a given amino acid sequence that can be flanked by from at least one, and up to as many as about 60, additional heterologous nucleotides at each of the 5′ and/or the 3′ end of the nucleic acid sequence encoding the given amino acid sequence. The heterologous nucleotides are not naturally found (i.e., not found in nature, in vivo) flanking the nucleic acid sequence encoding the given amino acid sequence as it occurs in the natural gene.

The invention is primarily directed to the use of fragments of full-length Axl and Mer proteins of the invention. The invention also encompasses fragments of the variants of the polypeptides described herein. As used herein, a fragment comprises at least 6 contiguous amino acids and includes any fragment of a full-length Axl or Mer protein described herein, and more preferably includes the entire extracellular domain of Axl or Mer or any portion thereof that retains the ability to bind to an Axl or Mer ligand. Fragments can be discrete (not fused to other amino acids or polypeptides) or can be within a larger polypeptide (as in a fusion protein of the present invention). Therefore, fragments can include any size fragment between about 6 amino acids and one amino acid less than the full length protein, including any fragment in between, in whole integer increments (e.g., 7, 8, 9 . . . 67, 68, 69 . . . 278, 279, 280 . . . amino acids).

Homologues of Axl or Mer proteins, including peptide and non-peptide agonists and antagonists of Axl or Mer (analogues), can be products of drug design or selection and can be produced using various methods known in the art. Such homologues can be referred to as mimetics. A mimetic refers to any peptide or non-peptide compound that is able to mimic the biological action of a naturally occurring peptide, often because the mimetic has a basic structure that mimics the basic structure of the naturally occurring peptide and/or has the salient biological properties of the naturally occurring peptide. Mimetics can include, but are not limited to: peptides that have substantial modifications from the prototype such as no side chain similarity with the naturally occurring peptide (such modifications, for example, may decrease its susceptibility to degradation); anti-idiotypic and/or catalytic antibodies, or fragments thereof; non-proteinaceous portions of an isolated protein (e.g., carbohydrate structures); or synthetic or natural organic molecules, including nucleic acids and drugs identified through combinatorial chemistry, for example. Such mimetics can be designed, selected and/or otherwise identified using a variety of methods known in the art. Various methods of drug design, useful to design or select mimetics or other therapeutic compounds useful in the present invention are disclosed in Maulik et al., 1997, Molecular Biotechnology: Therapeutic Applications and Strategies, Wiley-Liss, Inc.

Homologues can be produced using techniques known in the art for the production of proteins including, but not limited to, direct modifications to the isolated, naturally occurring protein, direct protein synthesis, or modifications to the nucleic acid sequence encoding the protein using, for example, classic or recombinant DNA techniques to effect random or targeted mutagenesis. For smaller peptides, chemical synthesis methods may be preferred. For example, such methods include well known chemical procedures, such as solution or solid-phase peptide synthesis, or semi-synthesis in solution beginning with protein fragments coupled through conventional solution methods. Such methods are well known in the art and may be found in general texts and articles in the area such as: Merrifield, 1997, Methods Enzymol. 289:3-13; Wade et al., 1993, Australas Biotechnol. 3(6):332-336; Wong et al., 1991, Experientia 47(11-12):1123-1129; Carey et al., 1991, Ciba Found Symp. 158:187-203; Plaue et al., 1990, Biologicals 18(3):147-157; Bodanszky, 1985, Int. J. Pept. Protein Res. 25(5):449-474; or H. Dugas and C. Penney, BIOORGANIC CHEMISTRY, (1981) at pages 54-92. For example, peptides may be synthesized by solid-phase methodology utilizing a commercially available peptide synthesizer and synthesis cycles supplied by the manufacturer. One skilled in the art recognizes that the solid phase synthesis could also be accomplished using the FMOC strategy and a TFA/scavenger cleavage mixture.

The polypeptides (including fusion proteins) of the invention can be purified to homogeneity. It is understood, however, that preparations in which the polypeptide is not purified to homogeneity are useful. The critical feature is that the preparation allows for the desired function of the polypeptide, even in the presence of considerable amounts of other components. Thus, the invention encompasses various degrees of purity. In one embodiment, the language “substantially free of cellular material” includes preparations of the polypeptide having less than about 30% (by dry weight) other proteins (i.e., contaminating protein), less than about 20% other proteins, less than about 10% other proteins, or less than about 5% other proteins.

According to the present invention, an isolated Axl or Mer protein, including a biologically active homologue or fragment thereof, has at least one characteristic of biological activity of a wild-type, or naturally occurring Axl or Mer protein. Biological activity of Axl or Mer and methods of determining the same have been described. A particularly preferred Axl or Mer protein for use in the present invention is an Axl or Mer protein variant that binds a ligand of Axl or Mer. Signaling function is not required for most of the embodiments of the invention and indeed, is not desired in the case of an Axl or Mer fusion protein that is an Axl or Mer inhibitor as described herein. In one aspect, the Axl or Mer protein binds to any ligand of naturally occurring Axl or Mer, including Gas6. In one aspect, the Axl or Mer protein binds to Protein S. In another aspect, the Axl or Mer protein preferentially binds to one Axl or Mer ligand as compared to another Axl or Mer ligand. In one aspect, the Axl or Mer protein binds to a TAM receptor, preferably sufficiently to inhibit the activation of the TAM receptor (e.g., such as by blocking or inhibiting the binding of a natural ligand to the TAM receptor and/or inhibiting receptor dimerization, trimerization or formation of any receptor-protein complex). In this aspect, ligand binding by the Axl or Mer protein can be retained or not retained. Most preferably, an Axl or Mer protein of the invention includes any Axl or Mer protein and preferably any Axl or Mer fusion protein with improved stability and/or half-life in vivo that is a competitive inhibitor of Axl or Mer (e.g., that preferentially binds to an Axl or Mer ligand as compared to an endogenous Axl or Mer cellular receptor). Such fusion proteins have been described in detail above.

Preferably, an Axl or Mer inhibitor of the invention, including an Axl or Mer fusion protein (e.g., an Axl-Fc fusion protein or a Mer-Fc fusion protein), binds to an Axl or Mer ligand with an equal or greater affinity as compared to the binding of the ligand to a naturally occurring Axl or Mer receptor tyrosine kinase (e.g., an Axl RTK or a Mer RTK expressed endogenously by a cell). In one embodiment, the Axl or Mer fusion protein inhibits the binding of an Axl or Mer ligand to a naturally occurring Axl or Mer receptor tyrosine kinase (or to a Tyro-3 receptor tyrosine kinase) and subsequent activation of the Axl or Mer RTK. For example, one can measure the Axl RTK and the Mer RTK activation using a phospho-Axl or phsopho-Mer analysis by Western blot. In one embodiment, binding of an Axl ligand to a naturally occurring Axl receptor tyrosine kinase is inhibited by at least 50%, at least 60%, at least 70%, at least 80%, at least 90%, or greater, using any suitable method of measurement of binding, as compared to an appropriate control. In another embodiment, binding of a Mer ligand to a naturally occurring Mer receptor tyrosine kinase is inhibited by at least 50%, at least 60%, at least 70%, at least 80%, at least 90%, or greater, using any suitable method of measurement of binding, as compared to an appropriate control.

Axl and Mer fusion proteins of the invention can, in some embodiments, be produced as chimeric proteins with additional proteins or moieties (e.g., chemical moieties) that have a second biological activity. For example, Axl or Mer fusion proteins, in addition to comprising the Axl or Mer protein and fusion partner as described above, may comprise a protein that has a biological activity that is useful in a method of the invention, such as a pro-apoptotic protein, in the case of treating a neoplastic disease. Alternatively, the additional protein portion of the chimera may be a targeting moiety, in order to deliver the Axl or Mer fusion protein to a particular in vivo site (a cell, tissue, or organ). Such additional proteins or moieties may be produced recombinantly or post-translationally, by any suitable method of conjugation.

Another embodiment of the present invention relates to the therapeutic use of novel anti-Mer or anti-Axl antibodies, and particularly, novel anti-human Axl or Mer antibodies, and even more particularly, novel anti-human Axl or Mer monoclonal antibodies (mAb) which lead to downregulation of the Axl or Mer proteins on the surface of neoplastic cells upon contact with cells displaying these transmembrane proteins. Also included in this embodiment are antigen-binding fragments of such antibodies. An exemplary monoclonal antibody of the present invention can detect, by any method (e.g., Western blot), the spectrum of Axl or Mer glycosylation states existing in normal human tissue and in human disease, or alternatively, selectively binds to a particular Axl or Mer glycoform and not to other Axl or Mer glycoforms.

Accordingly, one aspect of the invention is an anti-human Axl or Mer monoclonal antibody that can be used in the therapeutic methods of treating or preventing neoplastic disorders in a mammal as described above, alone or preferably in conjunction with an anti-cancer compound. Preferably, an antibody encompassed by the present invention includes any antibody that selectively binds to a conserved binding surface or epitope of an Axl or Mer protein, and preferably, to a conserved binding surface or epitope in the extracellular domain of the Axl or Mer protein. As used herein, an “epitope” of a given protein or peptide or other molecule is generally defined, with regard to antibodies, as a part of or a site on a larger molecule to which an antibody or antigen-binding fragment thereof will bind, and against which an antibody will be produced. The term epitope can be used interchangeably with the term “antigenic determinant,” “antibody binding site,” or “conserved binding surface” of a given protein or antigen. More specifically, an epitope can be defined by both the amino acid residues involved in antibody binding and also by their conformation in three dimensional space (e.g., a conformational epitope or the conserved binding surface). An epitope can be included in peptides as small as about 4-6 amino acid residues, or can be included in larger segments of a protein, and need not be comprised of contiguous amino acid residues when referring to a three dimensional structure of an epitope, particularly with regard to an antibody-binding epitope. Antibody-binding epitopes are frequently conformational epitopes rather than a sequential epitope (i.e., linear epitope), or in other words, an epitope defined by amino acid residues arrayed in three dimensions on the surface of a protein or polypeptide to which an antibody binds. As mentioned above, the conformational epitope is not comprised of a contiguous sequence of amino acid residues, but instead, the residues are perhaps widely separated in the primary protein sequence, and are brought together to form a binding surface by the way the protein folds in its native conformation in three dimensions.

As used herein, the term “selectively binds to” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), and flow cytometry.

One embodiment of the present invention includes an antibody or antigen binding fragment thereof that is a competitive inhibitor of the binding of an Axl or Mer ligand (e.g., Gas6 or Protein S) to an Axl or Mer receptor that is expressed by a particular cell or cell type. According to the present invention, a competitive inhibitor is an inhibitor (e.g., another antibody or antigen binding fragment or polypeptide) that binds to Axl or Mer that is expressed by a cell, and inhibits or blocks the binding of a natural Axl or Mer ligand (e.g., Gas6 or Protein S) to the Axl or Mer that is expressed by the cell. The antibody competitive inhibitor can also be defined by its ability to bind to Axl or Mer expressed by the cell at the same or similar epitope as another anti-Axl or anti-Mer antibody such that binding of the anti-Axl or anti-Mer antibody is inhibited. A competitive inhibitor may bind to the target with a greater affinity for the target than the Axl or Mer ligand. Competition assays can be performed using standard techniques in the art (e.g., competitive ELISA or other binding assays). For example, competitive inhibitors can be detected and quantitated by their ability to inhibit the binding of Axl or Mer to another, labeled anti-Axl or anti-Mer antibody, respectively.

Isolated antibodies of the present invention can include serum containing such antibodies, or antibodies that have been purified to varying degrees. Whole antibodies of the present invention can be polyclonal or monoclonal. Alternatively, functional equivalents of whole antibodies, such as antigen binding fragments in which one or more antibody domains are truncated or absent (e.g., Fv, Fab, Fab′, or F(ab)₂ fragments), as well as genetically-engineered antibodies or antigen binding fragments thereof, including single chain antibodies, humanized antibodies, fully human antibodies, antibodies that can bind to more than one epitope (e.g., bi-specific antibodies), or antibodies that can bind to one or more different antigens (e.g., bi- or multi-specific antibodies), may also be employed in the invention.

Limited digestion of an immunoglobulin with a protease may produce two fragments. An antigen binding fragment is referred to as an Fab, an Fab′, or an F(ab′)₂ fragment. A fragment lacking the ability to bind to antigen is referred to as an Fc fragment. An Fab fragment comprises one arm of an immunoglobulin molecule containing a L chain (V_{L}+C_{L} domains) paired with the V_{H} region and a portion of the C_{H} region (CH1 domain). An Fab′ fragment corresponds to an Fab fragment with part of the hinge region attached to the CH1 domain. An F(ab′)₂ fragment corresponds to two Fab′ fragments that are normally covalently linked to each other through a di-sulfide bond, typically in the hinge regions.

The C_{H} domain defines the isotype of an immunoglobulin and confers different functional characteristics depending upon the isotype. For example, μ constant regions enable the formation of pentameric aggregates of IgM molecules and α constant regions enable the formation of dimers.

Other functional aspects of an immunoglobulin molecule include the valency of an immunoglobulin molecule, the affinity of an immunoglobulin molecule, and the avidity of an immunoglobulin molecule. As used herein, affinity refers to the strength with which an immunoglobulin molecule binds to an antigen at a single site on an immunoglobulin molecule (i.e., a monovalent Fab fragment binding to a monovalent antigen). Affinity differs from avidity, which refers to the sum total of the strength with which an immunoglobulin binds to an antigen. Immunoglobulin binding affinity can be measured using techniques standard in the art, such as competitive binding techniques, equilibrium dialysis or BIAcore methods. As used herein, valency refers to the number of different antigen binding sites per immunoglobulin molecule (i.e., the number of antigen binding sites per antibody molecule of antigen binding fragment). For example, a monovalent immunoglobulin molecule can only bind to one antigen at one time, whereas a bivalent immunoglobulin molecule can bind to two or more antigens at one time, and so forth.

In one embodiment, the antibody is a bi- or multi-specific antibody. A bi-specific (or multi-specific) antibody is capable of binding two (or more) antigens, as with a divalent (or multivalent) antibody, but in this case, the antigens are different antigens (i.e., the antibody exhibits dual or greater specificity). For example, an antibody that selectively binds to Mer can be constructed as a bi-specific antibody, wherein the second antigen binding specificity is for a desired target, such as another cell surface marker on a target cell.

Antibodies of the present invention can include, but are not limited to, neutralizing antibodies, catalytic antibodies and blocking (binding) antibodies. According to the present invention, a neutralizing antibody is an antibody that reacts with an infectious agent (usually a virus) and destroys or inhibits its infectivity and virulence. A catalytic antibody is an antibody selected for its ability to catalyze a chemical reaction by binding to and stabilizing the transition-state intermediate. A blocking antibody is an antibody that binds to an antigen and blocks another antibody or agent from later binding to that antigen.

In one embodiment, antibodies of the present invention include humanized antibodies. Humanized antibodies are molecules having an antigen binding site derived from an immunoglobulin from a non-human species, the remaining immunoglobulin-derived parts of the molecule being derived from a human immunoglobulin. The antigen binding site may comprise either complete variable regions fused onto human constant domains or only the complementarity determining regions (CDRs) grafted onto appropriate human framework regions in the variable domains. Humanized antibodies can be produced, for example, by modeling the antibody variable domains, and producing the antibodies using genetic engineering techniques, such as CDR grafting (described below). A description various techniques for the production of humanized antibodies is found, for example, in Morrison et al. (1984) Proc. Natl. Acad. Sci. USA 81:6851-55; Whittle et al. (1987) Prot. Eng. 1:499-505; Co et al. (1990) J. Immunol. 148:1149-1154; Co et al. (1992) Proc. Natl. Acad. Sci. USA 88:2869-2873; Carter et al. (1992) Proc. Natl. Acad. Sci. 89:4285-4289; Routledge et al. (1991) Eur. J. Immunol. 21:2717-2725 and PCT Patent Publication Nos. WO 91/09967; WO 91/09968 and WO 92/113831.

In one embodiment, antibodies of the present invention include fully human antibodies. Fully human antibodies are fully human in nature. One method to produce such antibodies having a particular binding specificity includes obtaining human antibodies from immune donors (e.g., using EBV transformation of B-cells or by PCR cloning and phage display). In addition, and more typically, synthetic phage libraries have been created which use randomized combinations of synthetic human antibody V-regions. By selection on antigen, “fully human antibodies” can be made in which it is assumed the V-regions are very human like in nature. Phage display libraries are described in more detail below. Finally, fully human antibodies can be produced from transgenic mice. Specifically, transgenic mice have been created which have a repertoire of human immunoglobulin germline gene segments. Therefore, when immunized, these mice produce human like antibodies. All of these methods are known in the art.

Genetically engineered antibodies of the invention include those produced by standard recombinant DNA techniques involving the manipulation and re-expression of DNA encoding antibody variable and/or constant regions. Particular examples include, chimeric antibodies, where the V_{H} and/or V_{L} domains of the antibody come from a different source as compared to the remainder of the antibody, and CDR grafted antibodies (and antigen binding fragments thereof), in which at least one CDR sequence and optionally at least one variable region framework amino acid is (are) derived from one source and the remaining portions of the variable and the constant regions (as appropriate) are derived from a different source. Construction of chimeric and CDR-grafted antibodies are described, for example, in European Patent Applications: EP-A 0194276, EP-A 0239400, EP-A 0451216 and EP-A 0460617.

In one embodiment, chimeric antibodies are produced according to the present invention comprising antibody variable domains that bind to Axl or Mer and fused to these domains, a protein that serves as a second targeting moiety. For example, the targeting moiety can include a protein that is associated with the cell or tissue to be targeted or with a particular system in the animal.

Generally, in the production of an antibody, a suitable experimental animal, such as, for example, but not limited to, a rabbit, a sheep, a hamster, a guinea pig, a mouse, a rat, or a chicken, is exposed to an antigen against which an antibody is desired. Typically, an animal is immunized with an effective amount of antigen that is injected into the animal. An effective amount of antigen refers to an amount needed to induce antibody production by the animal. The animal's immune system is then allowed to respond over a pre-determined period of time. The immunization process can be repeated until the immune system is found to be producing antibodies to the antigen. In order to obtain polyclonal antibodies specific for the antigen, serum is collected from the animal that contains the desired antibodies (or in the case of a chicken, antibody can be collected from the eggs). Such serum is useful as a reagent. Polyclonal antibodies can be further purified from the serum (or eggs) by, for example, treating the serum with ammonium sulfate.

Monoclonal antibodies may be produced according to the methodology of Kohler and Milstein (Nature 256:495-497, 1975), or using the human B-cell hybridoma method, Kozbor, J., Immunol, 133:3001 (1984); Brodeur et al., Monoclonal Antibody Production Techniques and Applications, pp. 51-63 (Marcel Dekker, Inc., New York, 1987). For example, B lymphocytes are recovered from the spleen (or any suitable tissue) of an immunized animal and then fused with myeloma cells to obtain a population of hybridoma cells capable of continual growth in suitable culture medium. Hybridomas producing the desired antibody are selected by testing the ability of the antibody produced by the hybridoma to bind to the desired antigen. The hybridomas may be cloned and the antibodies may be produced by and then isolated from the hybridomas. A preferred method to produce antibodies of the present invention includes (a) administering to an animal an effective amount of a protein or peptide (e.g., an Axl or Mer protein or peptide including extracellular domains thereof) to produce the antibodies and (b) recovering the antibodies. As used herein, the term “monoclonal antibody” includes chimeric, humanized, and human forms of a monoclonal antibody. Monoclonal antibodies are often synthesized in the laboratory in pure form by a single clone (population) of cells. These antibodies can be made in large quantities and have a specific affinity for certain target antigens which can be found on the surface of cells.

In another method, antibodies of the present invention are produced recombinantly. For example, once a cell line, for example a hybridoma, expressing an antibody according to the invention has been obtained, it is possible to clone therefrom the cDNA and to identify the variable region genes encoding the desired antibody, including the sequences encoding the CDRs. From here, antibodies and antigen binding fragments according to the invention may be obtained by preparing one or more replicable expression vectors containing at least the DNA sequence encoding the variable domain of the antibody heavy or light chain and optionally other DNA sequences encoding remaining portions of the heavy and/or light chains as desired, and transforming/transfecting an appropriate host cell, in which production of the antibody will occur. Suitable expression hosts include bacteria, (for example, an E. coli strain), fungi, (in particular yeasts, e.g. members of the genera Pichia, Saccharomyces, or Kluyveromyces,) and mammalian cell lines, e.g. a non-producing myeloma cell line, such as a mouse NSO line, or CHO cells. In order to obtain efficient transcription and translation, the DNA sequence in each vector should include appropriate regulatory sequences, particularly a promoter and leader sequence operably linked to the variable domain sequence. Particular methods for producing antibodies in this way are generally well known and routinely used. For example, basic molecular biology procedures are described by Maniatis et al. (Molecular Cloning, Cold Spring Harbor Laboratory, New York, 1989); DNA sequencing can be performed as described in Sanger et al. (PNAS 74:5463, (1977)) and the Amersham International plc sequencing handbook; and site directed mutagenesis can be carried out according to the method of Kramer et al. (Nucl. Acids Res. 12, 9441, (1984)) and the Anglian Biotechnology Ltd. handbook. Additionally, there are numerous publications, including patent specifications, detailing techniques suitable for the preparation of antibodies by manipulation of DNA, creation of expression vectors and transformation of appropriate cells, for example as reviewed by Mountain A and Adair, J R in Biotechnology and Genetic Engineering Reviews (ed. Tombs, M P, 10, Chapter 1, 1992, Intercept, Andover, UK) and in the aforementioned European Patent Applications.

Alternative methods, employing, for example, phage display technology (see for example, U.S. Pat. No. 5,969,108, U.S. Pat. No. 5,565,332, U.S. Pat. No. 5,871,907, U.S. Pat. No. 5,858,657, U.S. Pat. No. 5,223,409; Fuchs et al. Bio/Technology, 9:1370-1372 (1991); or Griffiths et al. EMBO J., 12:725-734 (1993)) or the selected lymphocyte antibody method of U.S. Pat. No. 5,627,052 may also be used for the production of antibodies and/or antigen fragments of the invention, as will be readily apparent to the skilled individual.

### Compositions of Invention

Another embodiment of the invention relates to a composition comprising, consisting essentially of, or consisting of any of the Axl or Mer fusion proteins or Axl or Mer antibodies and particularly Axl or Mer monoclonal antibodies described herein. In one aspect of this embodiment, the composition further comprises a pharmaceutically acceptable carrier. In another aspect, the composition further comprises at least one therapeutic agent for the treatment of cancer. In another aspect, the composition further comprises an Axl-Fc, a Mer-Fc or a Tyro-3-Fc protein.

In the embodiments of the present invention including a composition or formulation (e.g., for therapeutic purposes), such compositions or formulations can include any one or more of the Axl and/or Mer inhibitors described herein, and may additionally comprise one or more pharmaceutical carriers or other therapeutic agents, including other therapeutic agents having anti-cancer activity in a mammal.

In one aspect, the Axl and/or Mer inhibitors of the invention can be formulated with a pharmaceutically acceptable carrier (including an excipient, diluent, adjuvant or delivery vehicle). The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the compound is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Common suitable pharmaceutical carriers are described in “Remington's Pharmaceutical Sciences” by E. W. Martin.

The compositions can be formulated for a particular type or route of delivery, if desired, including for parenteral, transmucosal, (e.g., orally, nasally or transdermally). Parental routes include intravenous, intra-arteriole, intramuscular, intradermal, subcutaneous, intraperitoneal, intraventricular and intracranial administration.

In another embodiment, the therapeutic compound or composition of the invention can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss: New York, pp. 353-365 (1989). To reduce its systemic side effects, this may be a preferred method for introducing the compound.

In yet another embodiment, the therapeutic compound can be delivered in a controlled release system. For example, a polypeptide may be administered using intravenous infusion with a continuous pump, in a polymer matrix such as poly-lactic/glutamic acid (PLGA), a pellet containing a mixture of cholesterol and the anti-amyloid peptide antibody compound (U.S. Pat. No. 5,554,601) implanted subcutaneously, an implantable osmotic pump, a transdermal patch, liposomes, or other modes of administration.

The pharmaceutical compositions of the invention may further comprise a therapeutically effective amount of another agent or therapeutic compound, preferably in respective proportions such as to provide a synergistic effect in the said prevention or treatment. Alternatively, the pharmaceutical compositions of the invention can be administered concurrently with or sequentially with another pharmaceutical composition comprising such other therapeutic agent or compound. A therapeutically effective amount of a pharmaceutical composition of the invention relates generally to the amount needed to achieve a therapeutic objective. For example, inhibitors and compositions of the invention can be formulated with or administered with (concurrently or sequentially), other chemotherapeutic agents or anti-cancer treatments, when it is desired to treat a neoplastic disease.

In one embodiment of the invention, an Axl fusion protein inhibitor (e.g., Axl-Fc) can be provided in a composition with or administered with a Mer fusion protein (e.g., Mer-Fc) or a Tyro-3 fusion protein (e.g., Tyro-3-Fc). A preferred Mer-Fc protein does not activate Axl. A preferred Axl-Fc protein does not activate Mer.

Another embodiment of the invention relates to the use of any of the Axl fusion proteins or compositions described herein in the preparation of a medicament for the treatment of cancer.

Each publication or patent cited herein is incorporated herein by reference in its entirety.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention.

### EXAMPLES

### Example 1

Diagnostic bone marrow samples from patients with B-cell ALL were obtained from Children's Oncology Group and Denver Children's Hospital and analyzed by Western blot and flow cytometry for the expression of MerTK (hMer). Nineteen E2A-PBX1 B-ALL (EP+) and 14 non-E2A-PBX1 B-ALL (EP−) patient samples were processed. All 19 EP+ samples had MerTK protein expression by Western blot and/or flow cytometry. Conversely, 1/14 EP− samples was weakly positive for MerTK protein expression by flow cytometry and Western blot. Quantitative RT-PCR showed a 7-324 fold increase in MerTK transcript in EP+ samples in comparison to EP− samples (data not shown).

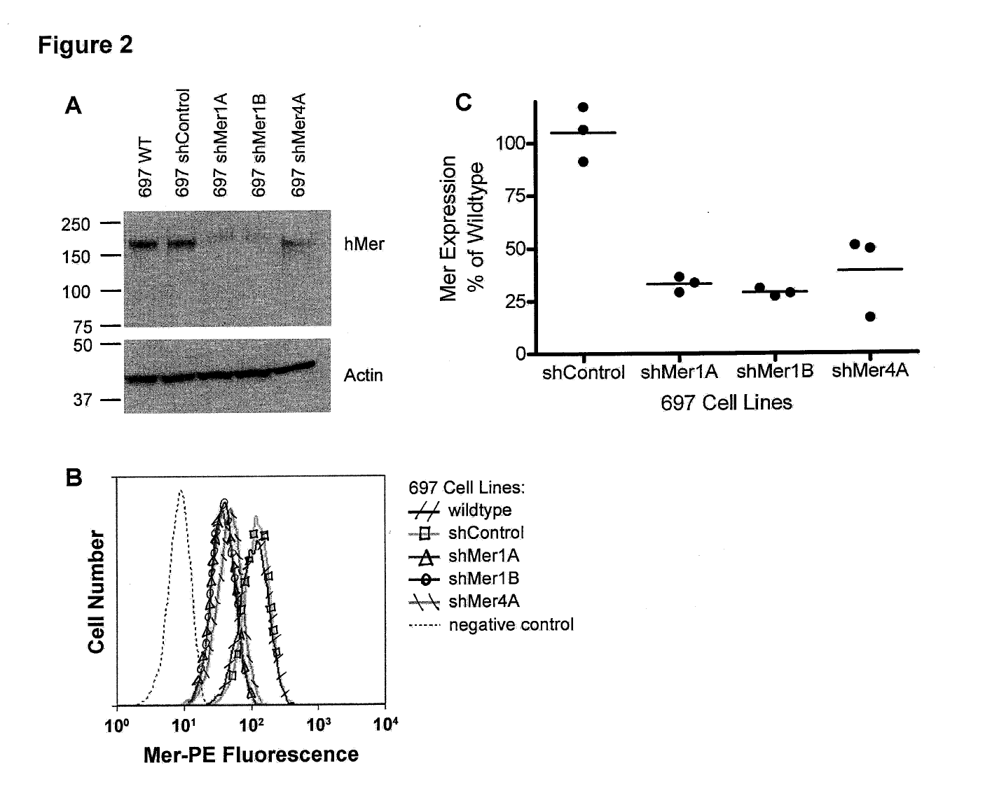

Short hairpin RNA (shRNA) was used to knockdown the expression of Mer and Axl in leukemia, lung adenocarcinoma and glioblastoma cell lines. Two Mer shRNA constructs (Mer 1 and Mer 4) have been tested for their ability to knockdown Mer expression in the human B-cell Acute Lymphoblastic Leukemia (ALL) cell line 697 which expresses the E2A-PBX1 fusion protein associated with the t(1;19) chromosomal rearrangement (Yeoh et al., 2002, Cancer Cell 1:133-143). Three clonal lines (shMer1A, shMer1B, and shMer4A) were established with two different shRNAs. Mer knockdown was confirmed by Western blot analysis of whole cell lysates (FIG. 2A) and flow cytometry (FIG. 2B). Densitometric analysis of semi-quantitative Western blots demonstrated significant (60-71%) knockdown in all three clonal lines (FIG. 2C). The non-silencing shRNA control (shControl) has also been successfully introduced into the 697 cells. This construct targets GFP and was selected as an appropriate non-silencing control construct since GFP is not present in these cells. This shRNA construct is in the same vector as the Mer shRNA constructs and engages the cellular RNAi machinery in a similar fashion. Actin is shown as a loading control.

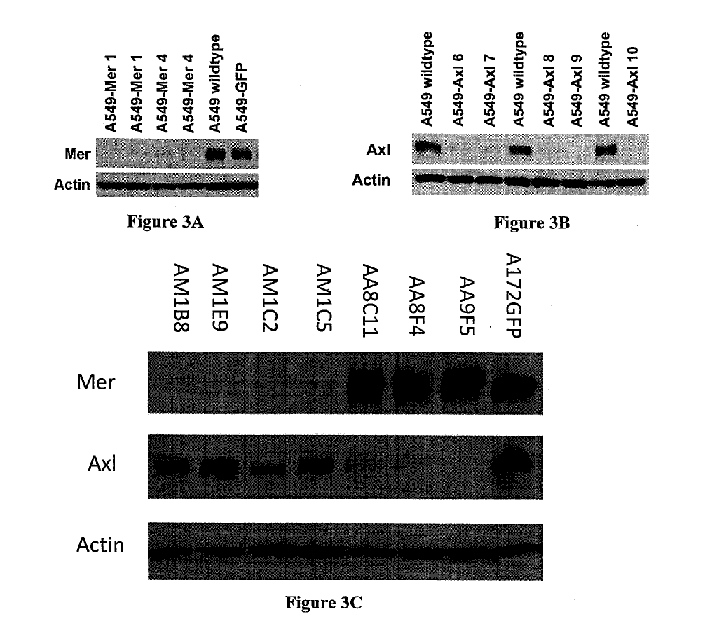

Similarly, seven shRNA constructs were used to knockdown Mer or Axl in the human lung adenocarcinoma cell line, A549. Western blot analysis of whole cell lysates demonstrating (FIG. 3A) Mer and (FIG. 3B) Axl knockdown are shown. Mer (FIG. 3A) and Axl (data not shown) levels were not affected by the non-silencing control (A549-GFP). The actin bands verify that equal amounts of total cellular protein were loaded.

Western blot analysis was conducted with whole cell lysates from A172 glioblastoma cells using different shRNA vectors. The results shown in FIG. 3C demonstrate Mer knockdown (lanes 1-4) and Axl knockdown (lanes 5-7). Mer and Axl levels were not affected by the non-silencing control (A172GFP, lane 8). The actin bands verify that equal amounts of total cellular protein were loaded.

### Example 2

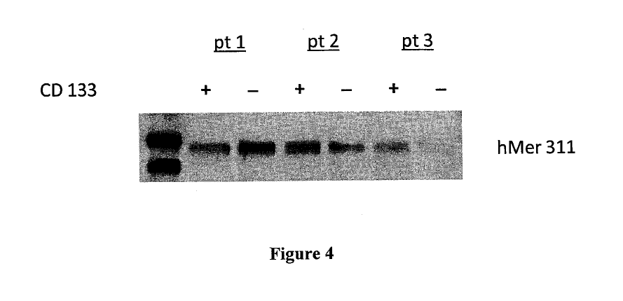

A glioblastoma stem cell population from three patients was examined for the presence of Mer receptor tyrosine kinase. As shown in FIG. 4, Western blot analysis of whole cell lysates from three separate patient samples demonstrate Mer expression in glioblastoma stem cell populations, as identified by the CD133 cell marker. Actin bands we used to verify that equal amounts of total cellular protein were loaded (data not shown).

### Example 3

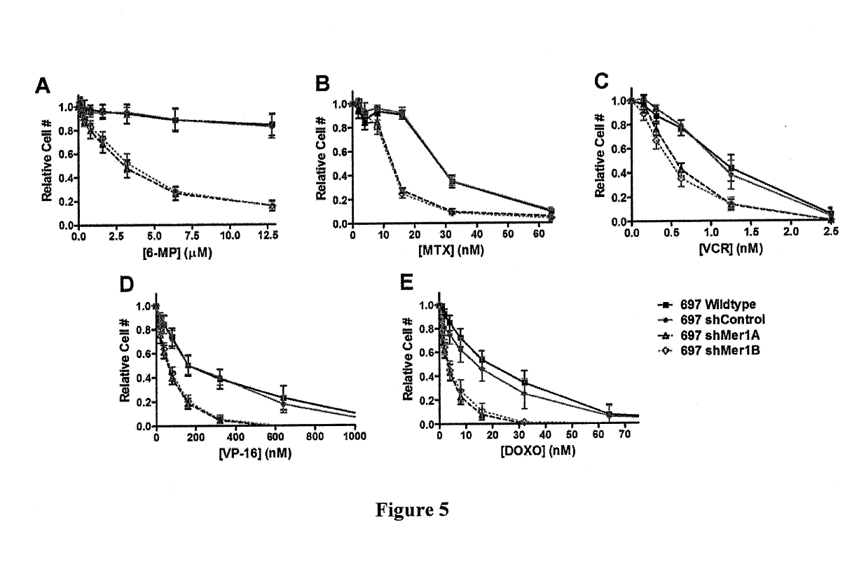

Inhibition of Mer expression results in increased chemosensitivity in 697 cells. As shown in FIG. 5, wild type, shControl, and Mer knockdown (shMer1A, shMer1B) 697 cells were treated with the indicated concentrations of: A) 6-mercaptopurine (6-MP); B) methotrexate (MTX); C) vincristine (VCR); D) etoposide (VP-16); or E) doxorubicin (DOXO) for 48 hours and relative cell numbers were determined. IC50 values were determined by non-linear regression of data from at least three independent experiments performed in triplicate, as shown in Table 1.

TABLE 1
IC50 values determined by non-linear regression of MTT assay data.

| | | 697 Human B-ALL Cell Lines | | | |
|---|---|---|---|---|---|
| | | Wild Type | shControl | shMer1A | shMer1B |
| 6-MP | IC₅₀ | >64 | >64 | 2.86 | 3.24 |
| (μM) | (99% CI) | ND | ND | (2.2-3.7) | (2.5-4.2) |
| | P < 0.01 | ND | — | ND | ND |
| MTX | IC₅₀ | 27.5 | 27.8 | 12.3 | 11.8 |
| (nM) | (99% CI) | (23.8-31.7) | (24.4-31.6) | (10.3-14.7) | (10.6-13.1) |
| | P < 0.01 | NS | — | * | * |
| VCR | IC₅₀ | 1.04 | 1.02 | 0.54 | 0.46 |
| (nM) | (99% CI) | (0.84-1.29) | (0.84-1.23) | (0.46-0.63) | (0.37-0.55) |
| | P < 0.01 | NS | — | * | * |
| VP-16 | IC₅₀ | 181 | 173 | 54.0 | 60.3 |
| (nM) | (99% CI) | (124-264) | (124-242) | (43.5-66.8) | (49.8-73.0) |
| | P < 0.01 | NS | — | * | * |
| DOXO | IC₅₀ | 16.7 | 11.1 | 2.83 | 3.26 |
| (nM) | (99% CI) | (12.4-22.4) | (7.17-17.2) | (2.20-3.64) | (2.44-4.35) |
| | P < 0.01 | NS | — | * | * |

Cumulative data from at least three independent experiments performed in triplicate were analyzed.
*Significance versus shControl is indicated by non-overlapping confidence intervals (99% CI). Significance could not be statistically evaluated for 6-MP treated cells because an IC50 was not reached for wild type or shControl cells.
Abbreviations are as follows: ND, Not determined; NS, Not significant; 6-MP, 6-mercaptopurine; MTX, methotrexate; VCR, vincristine; VP-16, etoposide; and DOXO, doxorubicin.

### Example 4

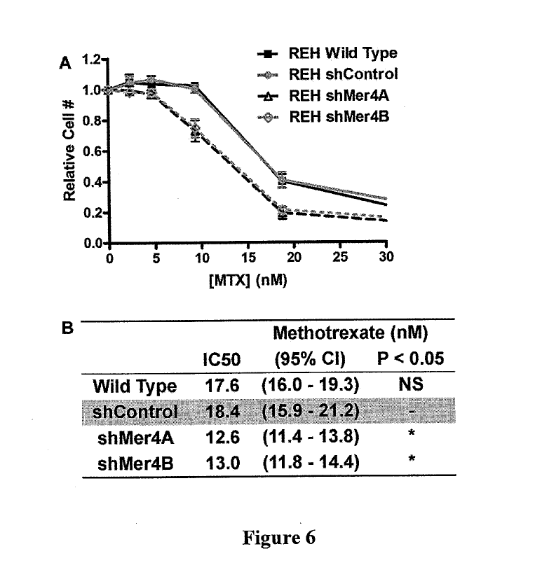

Inhibition of Mer expression results in increased chemosensitivity of REH cells. FIG. 6 shows cultures of REH wild type, shControl or Mer knockdown (shMer4A, shMer4B) cells treated with methotrexate (MTX) for 48 hours: A) relative cell numbers were determined by MTT assay and, B) IC₅₀ values were determined by non-linear regression of data from 3 independent experiments performed in triplicate. In comparison to shControl cells, Mer inhibition (shMer4A, shMer4B) resulted in significantly lower IC₅₀ values. Statistical differences versus shControl are indicated by non-overlapping confidence intervals (95% CI). No significant difference between wild type and shControl was observed.

### Example 5

Inhibition of Mer expression results in increased induction of apoptotic cell death in response to treatment with chemotherapeutic agents. Cultures of shControl or shMer1A 697 cells were exposed to 6-MP, VP-16, or medium only for 48 hours: A) Apoptotic and dead cells were identified by flow cytometric analysis of cells stained with YO-PRO®-1 and propidium iodide: Representative flow cytometry profiles; B) the percentages of apoptotic and dead cells are indicated in lower-right and upper-right quadrants, respectively. Mean values and standard errors derived from 3 independent experiments. Results were tested for significance using two-way ANOVA and Bonferroni posttests to compare shControl and shMer1A cells at equivalent drug concentrations. Significant differences in apoptotic cell populations are indicated by asterisks (* p<0.05, ** p<0.001). Significant differences in the number of dead cells were also observed († p<0.05, ‡ p<0.001).

### Example 6

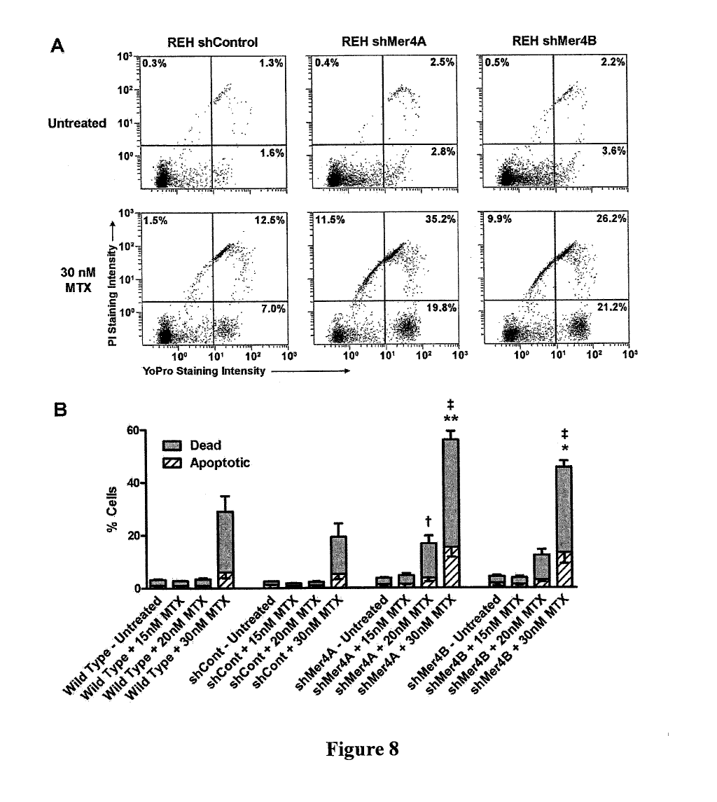

Inhibition of Mer leads to increased apoptotic cell death of REH cells in response to treatment with methotrexate. FIG. 8 shows cultures of REH wild type, shControl or Mer knockdown (shMer4A, shMer4B) cells exposed to methotrexate (MTX) or medium only for 48 hours. Apoptotic and dead cells were identified by flow cytometric analysis of cells stained with YO-PRO®-1 and propidium iodide: A) representative flow cytometry profiles. The percentage of apoptotic cells is indicated in the lower-right quadrant. The combined percentage of cells in the two upper quadrants indicates the fraction of dead cells. B) mean values and standard errors derived from 3 independent experiments. Results were tested for significance using two-way repeated measures ANOVA and Bonferroni posttests to compare shControl and Mer knockdown cells at equivalent drug concentrations. Significant differences in apoptotic cell populations are indicated by asterisks (* p<0.01, ** p<0.001). Significant differences in the number of dead cells were also observed († p<0.01, ‡‡ p<0.001). No significant differences between wild type and shControl cells were observed.

### Example 7

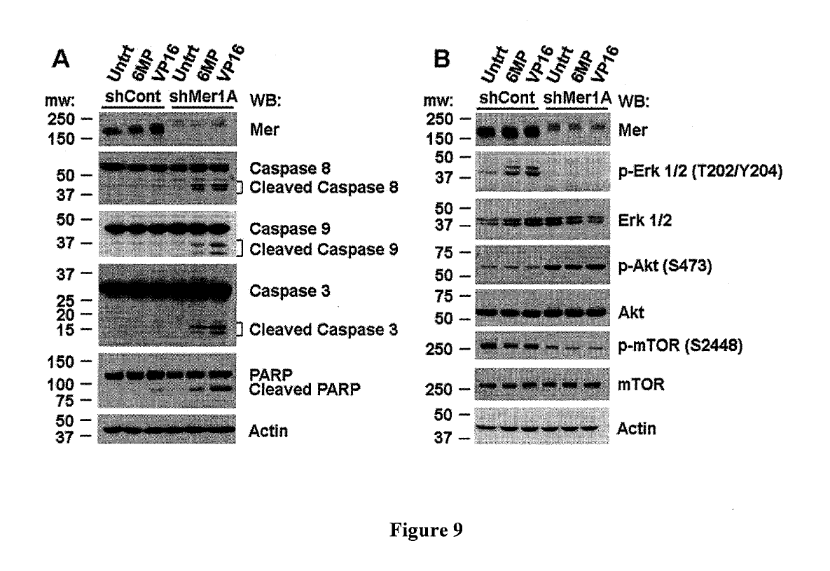

Knockdown of Mer inhibits survival signaling and promotes caspase cleavage. FIG. 9 shows control (shCont) and Mer knockdown (shMer1A) 697 cells exposed to 10 μM 6-MP, 150 nM VP-16, or medium only (Untrt) for 24 hours (A) or 40-60 minutes (B). Whole cell lysates were prepared and expression of the indicated proteins (p− denotes a phosphorylated protein) was determined by western blot analysis. Blots representative of three independent experiments are shown.

### Example 8

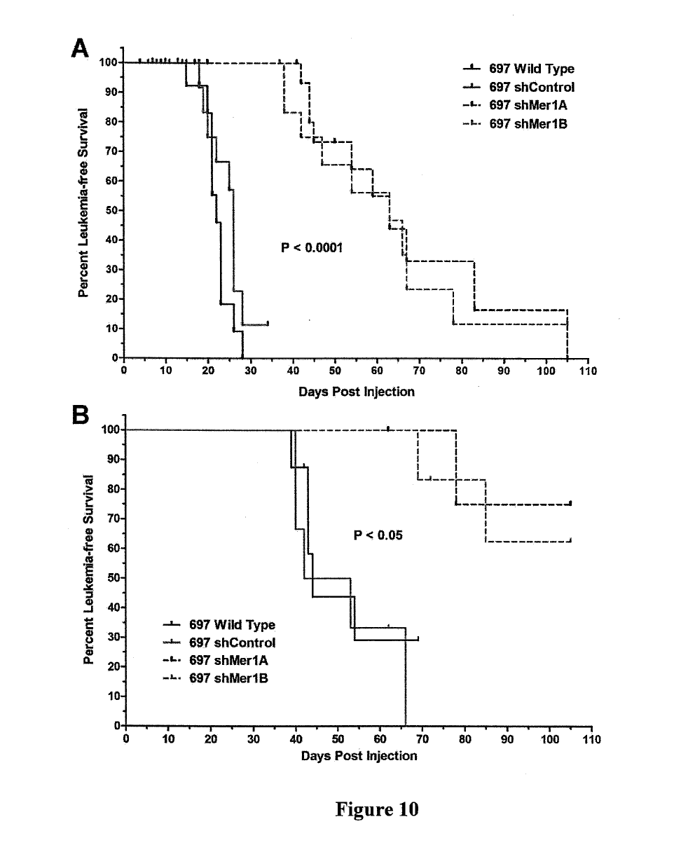

Mer inhibition significantly delays the onset of disease and improves leukemia-free survival in a mouse xenograft model of human leukemia. FIG. 10 shows the results of: A) sub-lethally irradiated NOD-SCID mice (10-26 animals per group) injected with 5×10⁶ cells of the indicated cell lines, or B) non-irradiated NOD-SCID mice (5-8 animals per group) injected with 5×10⁵ cells of the indicated cell lines. Ticks on the Kaplan-Meier survival curves indicate censored subjects (mice for which samples could not be obtained or did not have leukemia at time of death). Comparison of survival curves revealed a significant difference in leukemia-free survival with Mer inhibition (wild type or shControl vs. shMer1A or shMer1B, Log-rank test).

### Example 9

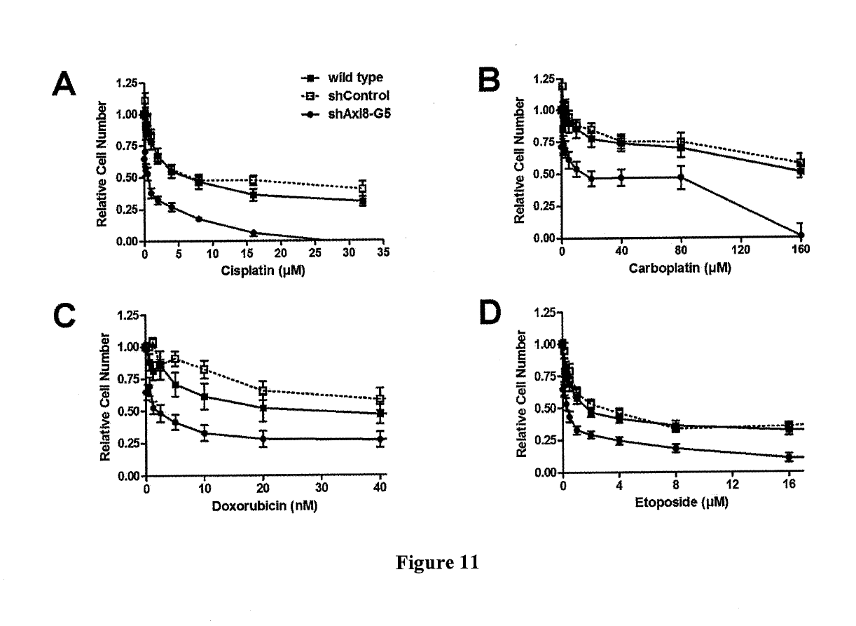

Inhibition of Axl expression results in increased chemosensitivity in A549 cells. FIG. 11 shows the relative cell numbers of wild type, shControl, and Axl knockdown (shAxl8-G5) A549 cells treated with the indicated concentrations of (A) cisplatin, (B) carboplatin, (C) doxorubicin, or (D) etoposide for 48 hours. IC₅₀ values were determined by non-linear regression of data from at least three independent experiments performed in triplicate as shown in Table 2. The shAxl9-D3 Axl knockdown clone is also more sensitive to cisplatin, doxorubicin, and etoposide.

TABLE 2
Cumulative data from at least three independent experiments
performed in triplicate were analyzed.

| | | A549 Human NSCLC Cell Lines | | | | |
|---|---|---|---|---|---|---|
| | | Wild Type | shControl | shAxl8-G5 | shAxl9-D3 | shMer1-G8 |
| Cispl | IC50 | 6.48 | 8.56 | 0.51 | 3.40 | 3.00 |
| (μM) | (95% CI) | (4.8-8.8) | (6.5-11.2) | (0.4-0.7) | (2.6-4.5) | (2.4-3.7) |
| | P < 0.05 | NS | — | * | * | * |
| Carbo | IC50 | >160 | >160 | 9.13 | ND | 93.4 |
| (μM) | (95% CI) | ND | ND | (5.8-14.4) | ND | (60.1-145) |
| | P < 0.05 | ND | — | ND | ND | ND |
| DOXO | IC50 | 34.9 | 69.2 | 2.1 | 13.0 | 71.2 |
| (nM) | (95% CI) | (18.0-67.7) | (45.5-105.4) | (1.0-4.2) | (8.6-19.7) | (31.2-162) |
| | P < 0.05 | NS | — | * | * | NS |
| VP-16 | IC50 | 2.20 | 3.32 | 0.33 | 1.01 | 3.84 |
| (μM) | (95% CI) | (1.58-3.05) | (2.47-4.46) | (0.24-0.46) | (0.70-1.46) | (2.78-5.30) |
| | P < 0.05 | NS | — | * | * | NS |

* Significance versus shControl is indicated by non-overlapping confidence intervals (95% CI). Significance could not be statistically evaluated for Carboplatin treated cells because an IC50 was not reached for wild type or shControl cells.
Abbreviations are as follows: ND, Not determined; NS, Not significant; Cispl, cisplatin; Carbo, carboplatin; DOXO, doxorubicin; VP-16, etoposide.

### Example 10

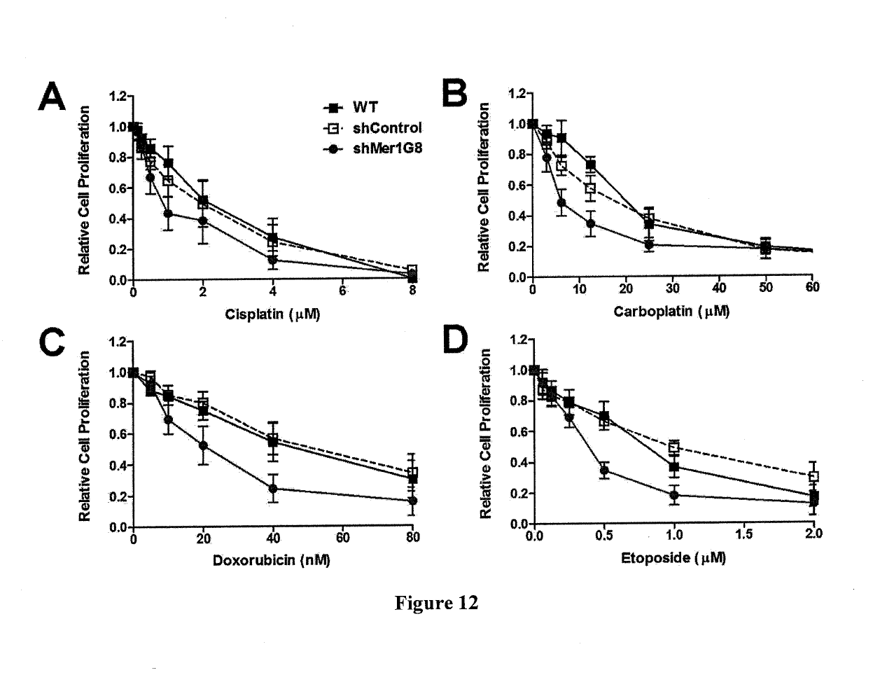

Inhibition of Mer expression results in increased chemosensitivity in A549 cells. FIG. 12 shows wild type, shControl, and Mer knockdown (Mer1-G8) A549 cells treated with the indicated concentrations of (A) cisplatin, (B) carboplatin, (C) doxorubicin, or (D) etoposide for 48 hours. The relative cell proliferation was determined via a BrdU incorporation ELISA. IC₅₀ values were determined by non-linear regression of data from at least three independent experiments performed in triplicate (see Table 3). Inhibition of Mer expression results in increased sensitivity of A549 cells to carboplatin, doxorubicin, and etoposide as shown in Table 3.

TABLE 3
IC50 values determined by non-linear regression of BrdU assay data.

| | | A549 Human NSCLC Cell Lines | | | | |
|---|---|---|---|---|---|---|
| | | Wild Type | shControl | shAxl8-G5 | shAxl9-D3 | shMer1-G8 |
| Cispl | IC50 | 2.1 | 1.7 | 2.7 | 2.3 | 1.0 |
| (μM) | (95% CI) | (1.5-2.8) | (1.1-2.4) | (1.5-4.8) | (1.3-4.1) | (0.7-1.4) |
| | P < 0.05 | NS | — | NS | NS | NS |
| Carbo | IC50 | 19.8 | 15.4 | 16.9 | 15.2 | 7.2 |
| (μM) | (95% CI) | (15.9-24.7) | (12.4-19.0) | (14.1-20.4) | (12.2-19.0) | (5.3-9.9) |
| | P < 0.05 | NS | — | NS | NS | * |
| DOXO | IC50 | 43.1 | 51.1 | 50.0 | 33.2 | 20.4 |
| (nM) | (95% CI) | (31.8-58.3) | (36.9-70.9) | (27.4-91.0) | (26.4-41.6) | (14.8-28.2) |
| | P < 0.05 | NS | — | NS | NS | * |
| VP-16 | IC50 | 0.72 | 0.91 | 1.40 | 0.59 | 0.37 |
| (μM) | (95% CI) | (0.55-0.95) | (0.69-1.22) | (0.83-2.36) | (0.49-0.71) | (0.30-0.46) |
| | P < 0.05 | NS | — | NS | NS | * |

Cumulative data from at least three independent experiments performed in triplicate were analyzed.
* Significance versus shControl is indicated by non-overlapping confidence intervals (95% CI).

### Example 11

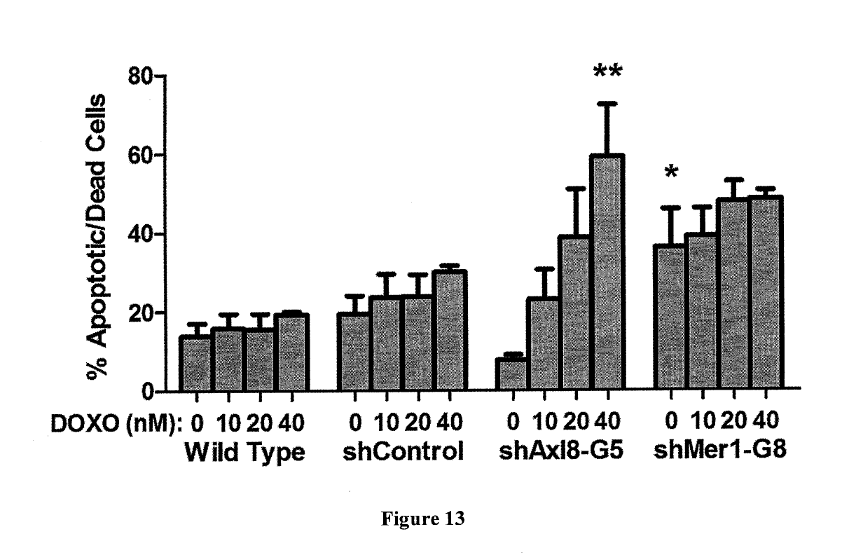

Inhibition of Axl results in increased induction of apoptosis in response to treatment with doxorubicin (DOXO). FIG. 13 shows the knockdown of Mer reduces survival of untreated A549 cells, and treatment with DOXO did not increase the level of cell death in Mer knockdown cells. Mean values and standard errors from at least three independent experiments are shown. * p<0.05 and ** p<0.001 vs. shControl, Bonferroni posttests.

### Example 12

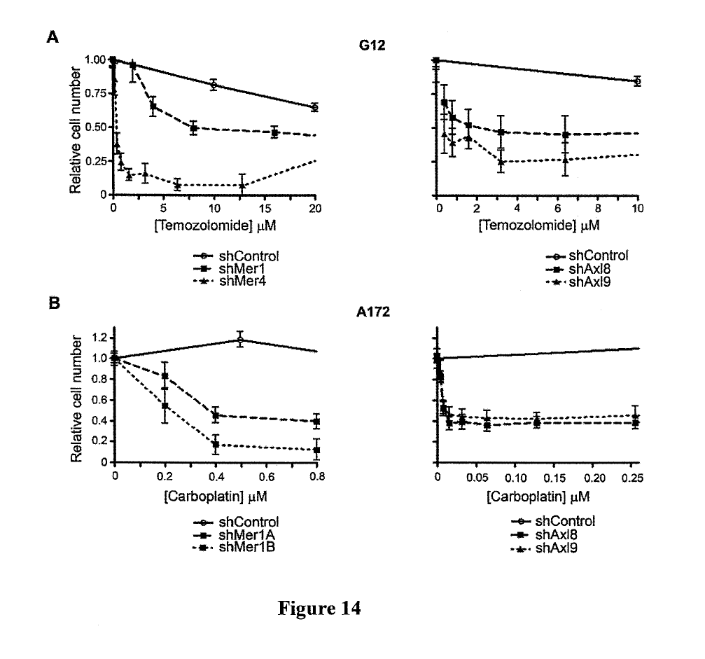

Chemosensitivity of astrocytoma cells as evaluated by MTT assay and the results are shown in FIG. 14. G12 control (shControl) and knockdown cells (shMer1 and shMer4; shAxl8 and shAxl9) were plated in 96-well plates at concentrations determined to allow linear growth over 3 days: A) cells were treated with varying concentrations of temozolomide and relative cell number was assessed 48 h after treatment by colorimetric assessment/MTT assay. Error bars denote experiments performed in triplicate. A172 control (shControl) and knockdown cells (shMer1A and shMer1B; shAxl8 and shAxl9) were plated in 96-well plates at concentrations determined to allow linear growth over 3 days: B) cells were treated with varying concentrations of carboplatin and relative cell number was assessed 48 h after treatment by colorimetric assessment/MTT assay. Error bars represent three independent experiments performed in triplicate.

Table 4 provides the IC50 with 95% CI for G12 and A172 control and Mer and Axl knockdown cells, in response to chemotherapy. In Table 4, ‘NR’ indicates an IC50 was never reached. ‘IND’ represents that that CI was indeterminate. The IC50 for each knockdown line was compared to the control line, and an ‘*’ represents that the comparison is statistically different while an ‘NS’ represents that there was no statistically significant difference because either the CI was indeterminate (as with the G12 shAxl9 line treated with carboplatin) or it overlapped with the shControl CI.

TABLE 4
The IC50 with 95% CI for G12 and A172 control and Mer and Axl knockdown cells in response to chemotherapy.

| | | | Temozolomide | | | Carboplatin | | | Vincristine | |
|---|---|---|---|---|---|---|---|---|---|---|
| | | | (μM) | | | (μM) | | | (nM) | |
| | | IC50 | (95% CI) | P < 0.05 | IC50 | (95% CI) | P < 0.05 | IC50 | (95% CI) | P < 0.05 |
| | shControl | 68.6 | (59.5-79.1) | | 47.9 | (29.48-77.98) | | 1.64 | (1.36-1.98) | |
| | shMer1 | 18.2 | (11.7-28.3) | * | 0.78 | (0.27-2.26) | * | 0.74 | (0.62-0.88) | * |
| G12 | shMer4 | 1 | (0.52-1.91) | * | 0.19 | (0.14-0.25) | * | 0.22 | (0.17-0.30) | * |
| | shAxl8 | 6.9 | (2.30-20.84) | * | 0.28 | (0.03-4.83) | * | 0.31 | (0.23-0.44) | * |
| | shAxl9 | 1.8 | (0.61-5.36) | * | 0.2 | IND | NS | 0.2 | (0.12-0.33) | * |
| | shControl | 51.4 | (40.6-65.0) | | NR | | | 2.54 | (1.91-3.37) | |
| | shMer1A | 21.3 | (15.0-30.0) | * | 0.82 | (0.36-1.80) | * | 1.25 | (0.96-1.63) | * |
| A172 | shMer1B | 7.7 | (5.5-10.7) | * | 0.21 | (0.13-0.35) | * | 0.54 | (0.42-0.66) | * |
| | shAxl8 | 27.6 | (19.3-39.5) | * | 0.035 | (0.02-0.08) | * | 0.68 | (0.53-0.86) | * |
| | shAxl9 | 38.9 | (26.6-56.6) | NS | 0.075 | (0.03-0.19) | * | 2.92 | (1.89-4.51) | NS |

‘NR’ indicates an IC50 was never reached. ‘IND’ represents that that CI was indeterminate. The IC50 for each knockdown line was compared to the control line, and an ‘*’ represents that the comparison is statistically different while an ‘NS’ represents that there was no statistically significant difference because either the CI was indeterminate (as with the G12 shAxl9 line treated with carboplatin) or it overlapped with the shControl CI.

### Example 13

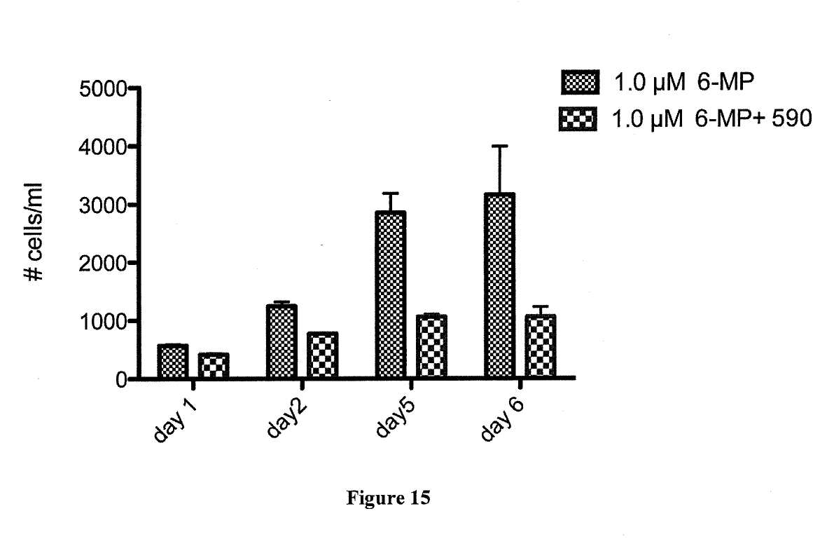

Downregulation of Mer on the surface of 697 leukemia cells after treatment with Mer monoclonal antibody 590 is shown in FIG. 15: A) following treatment of 697 cells with either 5 mg Mer monoclonal antibody or no antibody for 3-5 days, whole cell lysates were prepared and analyzed by SDS-PAGE using an anti-Mer monoclonal antibody; and B) Mer monoclonal antibody treatment synergizes with 6-MP in 697 leukemia cell line to decrease ability of leukemia cells to proliferate. Day 1=1st day following a 48-hour treatment window with chemotherapy +/−antibody.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed is:

1. A method of preventing or treating a neoplastic disorder in a mammal comprising inhibiting a receptor tyrosine kinase selected from the group consisting of Axl, Mer and Tyro-3 in the mammal.

2. (canceled)

3. The method of claim 1, wherein the neoplastic disorder is a cancer selected from the group consisting of glioma, gliosarcoma, anaplastic astrocytoma, medulloblastoma, lung cancer, small cell lung carcinoma, cervical carcinoma, colon cancer, rectal cancer, chordoma, throat cancer, Kaposi's sarcoma, lymphangiosarcoma, lymphangioendotheliosarcoma, colorectal cancer, endometrium cancer, ovarian cancer, breast cancer, pancreatic cancer, prostate cancer, renal cell carcinoma, hepatic carcinoma, bile duct carcinoma, choriocarcinoma, seminoma, testicular tumor, Wilms' tumor, Ewing's tumor, bladder carcinoma, angiosarcoma, endotheliosarcoma, adenocarcinoma, sweat gland carcinoma, sebaceous gland sarcoma, papillary sarcoma, papillary adenosarcoma, cystadenosarcoma, bronchogenic carcinoma, medullary carcinoma, mastocytoma, mesotheliorma, synovioma, melanoma, leiomyosarcoma, rhabdomyosarcoma, neuroblastoma, retinoblastoma, oligodentroglioma, acoustic neuroma, hemangioblastoma, meningioma, pinealoma, ependymoma, craniopharyngioma, epithelial carcinoma, embryonal carcinoma, squamous cell carcinoma, base cell carcinoma, fibrosarcoma, myxoma, myxosarcoma, liposarcoma, chondrosarcoma, osteogenic sarcoma, leukemia, and the metastatic lesions secondary to these primary tumors.

4. (canceled)

5. (canceled)

6. (canceled)

7. (canceled)

8. (canceled)

9. The method of claim 1, wherein the inhibiting comprises administering a fusion protein comprising at least a portion of the Axl extracellular domain, or the Mer extracellular domain fused to the Fc region of a human immunoglobulin to the mammal.

10. The method of claim 1, wherein the inhibiting comprises administering a small molecule inhibitor of Mer or Axl tyrosine kinase that prevents tyrosine kinase activitation.

11. The method of claim 1, wherein the inhibiting comprises administering an antibody that results in the downregulation of at least one of Axl and Mer tyrosine kinases to the mammal.

12. (canceled)

13. (canceled)

14. (canceled)

15. The methods of claim 1, wherein the inhibiting comprises the administration of a chemotherapeutic drug to the mammal concurrent to the inhibition of the receptor tyrosine kinase.

16. (canceled)

17. (canceled)

18. The methods of claim 15, wherein the chemotherapeutic drug is administered within two weeks of an inhibitor of the receptor tyrosine kinase.

19. The methods of claim 15, wherein the chemotherapeutic drug is selected from the group consisting of busulfan, thiotepa, chlorambucil, cyclophosphamide, estramustine, ifosfamide, mechloretharmine, melphalan, uramustine, carmustine, lomustine, streptozocin, dacarbazine, procarbazine, temozolamide, cisplatin, carboplatin, oxaliplatin, satraplatin, picoplatin, methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, fluorouracil, gemcitabine, troxacitabine, bleomycin, dactinomycin, mithramycin, mitomycin, mitoxantrone, porfiromycin, daunorubicin, daunorubicin, doxorubicin, liposomal doxorubicin, epirubicin, idarubicin, valrubicin, L-asparaginase, PEG-L-asparaginase, paclitaxel, docetaxel, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifene, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, lenalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, ¹³¹I-tositumomab, interferon-α₂ₐ, interferon-α_{2b}, aldesleukin, denileukin diftitox, and oprelvekin.

20. The methods of claim 15, wherein the chemotherapeutic drug is selected from the group consisting of 6-mercaptopurine, etoposide, adriamycin, vincristine and methotrexate.

21. (canceled)

22. A method of overcoming resistance of a neoplastic cell to a chemotherapeutic drug comprising inhibiting a receptor tyrosine kinase of the cell selected from the group consisting of Axl, Mer and Tyro-3.

23. (canceled)

24. (canceled)

25. (canceled)

26. (canceled)

27. The methods of claim 22, wherein the inhibiting comprises contacting the neoplastic cell with a fusion protein comprising a Fc region of a human antibody fused with a protein selected from the group consisting of at least a portion of an extracellular domain of Axl receptor tyrosine kinase receptor and at least a portion of an extracellular domain of Mer receptor tyrosine kinase receptor.

28. (canceled)

29. (canceled)

30. (canceled)

31. A method of treating cancer in a mammal comprising:
a. administering to the mammal a fusion protein comprising a Fc region of a human antibody fused with a protein selected from the group consisting of at least a portion of an extracellular domain of Axl receptor tyrosine kinase receptor and at least a portion of an extracellular domain of Mer receptor tyrosine kinase receptor; and,
b. administering to the mammal a chemotherapeutic drug selected from the group consisting of 6-mercaptopurine, etoposide, adriamycin, vincristine and methotrexate.

32. The method of claim 31, wherein the chemotherapeutic drug is administered to the mammal within one to three weeks of administering the fusion protein to the mammal.

33. A method of treating cancer in a mammal comprising:
a. administering to the mammal an antibody that recognizes an epitope on the extracellular domain of at least one of an Axl and Mer receptor tyrosine kinase; and,
b. administering to the mammal a chemotherapeutic drug selected from the group consisting of 6-mercaptopurine, etoposide, adriamycin, vincristine and methotrexate.

34. The method of claim 33, wherein the chemotherapeutic drug is administered to the mammal within one week (it is hard to know the timeline—it is possible that an antibody would inhibit longer than a week, as some of our studies suggest. Could we state within one week or one to three weeks?) of administering the antibody to the mammal.

35-49. (canceled)


SEQUENCE LISTING (2 sequences)

SEQ ID NO: 1 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 2 - protein, 999 aa, Homo sapiens
MGPAPLPLLLGLFLPALWRRAITEAREEAKPYPLFPGPFPGSLQTDHTPLLSLPHASGYQPALMFSPTQPGRPHTGNVAIPQVTSVESKPLPPLAFKHTVGHIILSEHKGVKFNCSISVPNIYQDTTISWWKDGKELLGAHHAITQFYPDDEVTAIIASFSITSVQRSDNGSYICKMKINNEEIVSDPIYIEVQGLPHFTKQPESMNVTRNTAFNLTCQAVGPPEPVNIFWVQNSSRVNEQPEKSPSVLTVPGLTEMAVFSCEAHNDKGLTVSKGVQINIKAIPSPPTEVSIRNSTAHSILISWVPGFDGYSPFRNCSIQVKEADPLSNGSVMIFNTSALPHLYQIKQLQALANYSIGVSCMNEIGWSAVSPWILASTTEGAPSVAPLNVTVFLNESSDNVDIRWMKPPTKQQDGELVGYRISHVWQSAGISKELLEEVGQNGSRARISVQVHNATCTVRIAAVTRGGVGPFSDPVKIFIPAHGWVDYAPSSTPAPGNADPVLIIFGCFCGFILIGLILYISLAIRKRVQETKFGNAFTEEDSELVVNYIAKKSFCRRAIELTLHSLGVSEELQNKLEDVVIDRNLLILGKILGEGEFGSVMEGNLKQEDGTSLKVAVKTMKLDNSSQREIEEFLSEAACMKDFSHPNVIRLLGVCIEMSSQGIPKPMVILPFMKYGDLHTYLLYSRLETGPKHIPLQTLLKFMVDIALGMEYLSNRNFLHRDLAARNCMLRDDMTVCVADFGLSKKIYSGDYYRQGRIAKMPVKWIAIESLADRVYTSKSDVWAFGVTMWEIATRGMTPYPGVQNHEMYDYLLHGHRLKQPEDCLDELYEIMYSCWRTDPLDRPTFSVLRLQLEKLLESLPDVRNQADVIYVNTQLLESSEGLAQGSTLAPLDLNIDPDSIIASCTPRAAISVVTAEVHDSKPHEGRYILNGGSEEWEDLTSAPSAAVTAEKNSVLPGERLVRNGVSWSHSSMLPLGSSLPDELLFADDSSEGSEVLM